(19) United States
(12) Patent Application Publication
(10) Pub. No.: US 2012/0317675 A1
(43) Pub. Date: Dec. 13, 2012

(54) ULTRA-LOW TRYPSIN INHIBITOR SOYBEAN

(75) Inventor: John A. Schillinger, Cumming, IA (US)

(73) Assignee: SCHILLINGER GENETICS, INC., West Des Moines, IA (US)

(21) Appl. No.: 13/579,087

(22) PCT Filed: Mar. 3, 2011
(86) PCT No.: PCT/US2011/027035

§ 371 Date: Aug. 15, 2012

Related U.S. Application Data
(60) Provisional application No. 61/310,233, filed on Mar. 3, 2010.
(60) Provisional application No. 61/314,919, filed on Mar. 17, 2010.

(51) Int. Cl. A01H 5/10; A23L 2/00; A23K 1/14; A23L 1/20; A23G 3/00; C12Q 1/68; C12N 5/04; C07K 14/415; C11B 1/00; A01H 1/02; C12N 15/82; A01H 5/00; A01H 5/12; A01H 5/06; A01H 5/02; A01H 5/04; A23L 1/39

(52) U.S. Cl. 800/260; 426/589; 426/590; 426/599; 426/630; 426/634; 435/6.11; 435/415; 530/378; 554/9; 800/278; 800/300; 800/301; 800/302; 800/312

(57) ABSTRACT

Soybeans (Glycine max) possessing a novel genetic allele for the production of reduced trypsin inhibitor in seeds is provided. Such alleles can be readily transferred to other soybean lines and cultivars. In a preferred embodiment a soybean plant possesses the combined presence of the Kunitz allele along with the SG-ULTI mutant allele, the combination of which was found to produce an ultra-low trypsin inhibitor phenotype in the resulting progeny seeds of a cross of a Kunitz allele into the 435.TCS background. A seed or seed product is made possible in this instance that is particularly well suited for consumption without extensive processing to remove trypsin inhibitor. The invention also relates to soybean seeds and plants containing the SG-ULTI mutant allele, and to methods for producing a soybean plant containing the SG-ULTI mutant allele produced by crossing a soybean plant containing the SG-ULTI mutant allele with itself or another soybean variety

### CROSS-REFERENCE TO RELATED APPLICATION

This application claims priority to both U.S. provisional Patent Application No. 61/310,233 filed Mar. 3, 2010, and U.S. provisional Patent Application No. 61/314,919, filed Mar. 17, 2010, which are incorporated herein by reference in their entirety.

### FIELD OF THE INVENTION

The present invention relates generally to the field of plant breeding and molecular biology. The invention further relates to agronomically elite soybean varieties with reduced trypsin inhibitor content and materials and methods for making such plants. All publications cited in this application are herein incorporated by reference.

Trypsin is an important digestive enzyme, particularly in certain species where ancillary enzymes, such as pepsin and chymotrypsin are present in relatively small amounts, or are absent. From an economic standpoint, the most important of these species are chickens, pigs, and calves (when the calves are sufficiently young that they have not developed a fully mature digestive system). In such animals, in particular, if the enzyme trypsin is in some way impaired in its functioning, there are a number of deleterious results. First, any food which is ingested by the animal is lowered in nutritive value because of a directly impaired capacity to digest it. Second, even in animals which contain other digestive enzymes in addition to trypsin, trypsin normally activates some of these enzymes and allows their participation in the process. A deficiency in trypsin thus results in a concomitant deficiency in these enzymes. Finally, in response to a perceived lack of adequate trypsin, the pancreas is induced to release more trypsin than it is easily capable of releasing, resulting in an “overwork” condition called pancreatic hypertrophy, which at best, results in morbidity and at worst, in death.

Kunitz trypsin inhibitor is an anti-nutritional and allergenic factor in soybeans that interferes with digestion and absorption of proteins when present in a diet. Genetic and biochemical studies of Kunitz trypsin inhibitor production in soybean lines have been carried out (e.g. de Moraes et al., 2006; Natarajan et al., 2006), and three related genes have been identified, with KTI3 encoding the predominant Kunitz trypsin inhibitor protein in cultivated soybean genotypes (Natarajan et al., 2006). Some specific DNA markers associated with loss of Kunitz production in certain soybean lines have been reported (de Moraes et al., 2006).

The Kunitz phenotype refers to a specific trypsin inhibitor and is responsible for a reduction in total trypsin inhibition (measured in TIU, trypsin inhibitor units) by roughly a third the level of commercially available soybeans. The unique phenotype of the instant application is an additional, stepwise, reduction in total trypsin inhibitor. It is likely that this reduction is the response to a mutation or mutations in other trypsin inhibitors, such as the Bowman-Burk trypsin inhibitors.

The Bowman-Birk trypsin inhibitors represent a group of soybean trypsin inhibitors that are genetically distinct from the Kunitz trypsin inhibitors. There are thought to be 6 to 10 different genes belonging to the Bowman-Birk class of inhibitors in soybeans, some mutants of which have been investigated (e.g., Livingstone, et al., Plant Mol. Biol., 64:397-408 (2007) The Bowman-Birk inhibitors appear to make up most of the remaining 65-70% of trypsin inhibitor activity not accounted for by the Kunitz trypsin inhibitors.

Trypsin inhibition is an insurmountable problem when the ingested foodstuff contains large quantities of soybean materials which have not been subjected to proper treatment to destroy a soybean trypsin inhibitor which is capable of binding the endogenous trypsin in the animal ingesting the foodstuff, and in preventing it from carrying out its normal function. Hence, animal foods which are largely soybean based are currently treated by “cooking” to inactivate this protein. In conventional soy processing, the soybeans are dehulled using a wet process, wherein the water content, however, is purposely limited in order to reduce waste weight and in order to prevent interference with subsequent processing steps. The hulled soybeans are then extracted with hexane to remove the soybean oil for commercial use. After the hexane extraction, the soybean mulch is heated to inactivate the soybean trypsin inhibitor.

This inactivation process is conducted at considerable expense, and with imperfect results. The heating produces a decline in soybean trypsin inhibitor content. Therefore, after a time period which is optimum for the particular preparation in question, further heating becomes uneconomical and counterproductive, even though additional amounts of soybean trypsin inhibitor would be thereby inactivated. The resulting processed soybean meal is then used in animal feeds in a variety of forms, and is reduced in soybean trypsin inhibitor but still contains residual amounts.

The most common use of this preparation is as a feed additive which is added to other carbohydrate sources used for livestock feeding, the most important livestock types being pigs and chickens, as well as newborn calves. However, as fed to calves, the preparation is more commonly used as a milk replacement by suspending the preparation in a liquid before feeding. This is formulated either as a solid which may subsequently be made up in liquid form by the livestock raiser, or as a liquid concentrate which is diluted before feeding. Although the constituency is smaller in number, soybean preparations are also used as feeding supplements for human infants, particularly those who exhibit intolerance for milk products.

The problem of trypsin inhibition has also been studied from a purely research viewpoint. It is known that soybean trypsin inhibitor reacts with bovine trypsin by specifically binding to the reactive site of trypsin. The soybean trypsin inhibitor is hydrolyzed at the interface due to the action of the inhibited trypsin itself (Laskowski and Sealock, Enzymes, 3rd edition, 375 (1971); Finkenstadt et al., Proceedings of the International Conference of Proteinase Inhibitors, Second, 389 (1974)). The mechanism of this inhibition is reasonably well understood (Mattis and Laskowski, Biochemistry 12: 2239 (1973); Ruhlmann et al., Journal of Molecular Biology, 77: 417 (1973); Huber et al., Journal of Molecular Biology, 89: 73 (1974), and Sweet et al., Biochemistry 13: 4212 (1974)). It appears that a fairly tight complex is formed between the inhibitor and the trypsin.

Incubation of catalytic amounts of trypsin with soybean trypsin inhibitor results in specific hydrolysis of a single peptide bond, the reactive site. This hydrolysis leads to the establishment of an equilibrium between virgin (reactive site peptide bond intact) and modified (reactive site peptide bond hydrolyzed) inhibitor. Both virgin and modified soybean trypsin inhibitor are inhibitors of trypsin, so this hydrolysis will not by itself inactivate soybean trypsin inhibitor. Although trypsin can catalyze the conversion of virgin to modified soybean trypsin inhibitor, it does so at such a slow rate (several days at neutral pH) that trypsin cannot be used effectively to inactivate soybean trypsin inhibitor.

### SUMMARY OF THE INVENTION

The present invention relates to a method for overcoming trypsin inhibition by soybean trypsin inhibitors in soy based foodstuffs. In one aspect of the present invention, novel soybean plants are generated having an ultra-low trypsin inhibitor activity and content. These new soybean plants result as the hybrid progeny of a cross between parental soybean lines having the Kunitz allele of trypsin inhibitor gene and the novel SG-ULTI mutant allele of a non-Kunitz gene.

Accordingly, it is one aspect of the present invention to provide a simple, effective, and inexpensive way to eliminate the problem of soybean trypsin inhibitor in soybean based food and food supplements which are commonly prepared from soybeans or soybean meal. The factors of the nutritive qualities of such foodstuffs and their cost of preparation are significant in determining their commercial success and therefore commercial availability.

It is another aspect of the invention to provide an animal with protection against ingested soybean trypsin inhibitor by furnishing food products that have an inherently low amount of such trypsin inhibitors as constituents of those food products.

A further aspect of the present invention is a method for overcoming trypsin inhibition by soybean trypsin inhibitor in soy-based foodstuffs. In another aspect of the invention, soybean plants are produced as the progeny of crossing a soybean line that has a mutation in the Kunitz trypsin inhibitor (Kunitz) gene, and thereby expressing the reduced trypsin inhibitor Kunitz phenotype, with a soybean line that has an allele that is not in the Kunitz gene, that also produces trypsin inhibitor activity that is lower than the Kunitz phenotype, referred to as the SG-ULTI phenotype. The resulting progeny plants have levels of trypsin inhibitor activity that are lower than either parent.

The present invention unexpectedly provides a reduced content of trypsin inhibitor, below even the content of lines containing the Kunitz mutation, and offers a solution to the problems caused by the presence of soybean trypsin inhibitor from desirability of use of soybean as a food.

Accordingly, it is one aspect of the present invention to provide a simple, effective, and inexpensive way to eliminate the problem of soybean trypsin inhibitor in soybean based foodstuffs.

It is another aspect of the invention to provide an animal with protection against ingested soybean trypsin inhibitor by furnishing foodstuffs or feeds that possess highly reduced soybean trypsin inhibitor content.

Another aspect of the present invention relates to a soybean mutant allele, designated “SG-ULTI”. The present invention also relates to soybean seed, a soybean plant and a soybean cultivar containing the SG-ULTI mutant allele. A further aspect of the invention further provides plants, seeds, and other plant parts such as pollen and ovules containing the mutant allele. In addition, another aspect of the present invention is directed to transferring the SG-ULTI mutant allele to other soybean cultivars and is useful for producing soybean cultivars and novel types with the SG-ULTI mutant allele trait.

Another aspect of the present invention also provides methods for introducing the SG-ULTI mutant allele into soybean plants by crossing a soybean plant which lacks the SG-ULTI mutant allele with a soybean plant that has the SG-ULTI mutant allele, selfing the resulting generations and then selecting the plants exhibiting one or more desired characteristics.

In another aspect, the invention provides a method for producing soybean seed comprising crossing a first plant parent with a second plant parent and harvesting the resultant soybean seed, wherein either one or both parents contain the SG-ULTI mutant allele.

In another aspect, the present invention provides for single gene converted plants containing the SG-ULTI mutant allele. The desired single transferred gene may preferably be a dominant or recessive allele. Preferably, the single transferred gene will confer such traits as herbicide resistance, insect resistance, resistance for bacterial, fungal, or viral disease, male fertility, male sterility, enhanced nutritional quality, and industrial usage. The single gene may be a naturally occurring soybean gene or a transgene introduced through genetic engineering techniques.

In another aspect, the present invention provides regenerable cells for use in tissue culture of a soybean plant containing the SG-ULTI mutant allele. The tissue culture will preferably be capable of regenerating plants having the physiological and morphological characteristics of the foregoing soybean plant, and of regenerating plants having substantially the same genotype as the foregoing soybean plant. Preferably, the regenerable cells in such tissue cultures will be embryos, protoplasts, meristematic cells, callus, pollen, leaves, anthers, pistils, roots, root tips, flowers, seeds, panicles or stems. Still further, the present invention provides soybean plants regenerated from the tissue cultures of the invention.

Another aspect of the invention relates to any soybean seed or plant having the SG-ULTI mutant allele.

Other aspects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DEFINITIONS

In the description and tables which follow, a number of terms are used. In order to provide a clear and consistent understanding of the specification and claims, the following definitions are provided:

Agronomically Elite: As used herein, means a soybean plant having improved traits such as seed yield, emergence, vigor, vegetative vigor, disease resistance, seed set, standability, and threshability which allows a producer to harvest a product of commercial significance.

Allele: Any of one or more alternative forms of a gene locus, all of which relate to a trait or characteristic. In a diploid cell or organism, the two alleles of a given gene occupy corresponding loci on a pair of homologous chromosomes.

Backcrossing: A process in which a breeder repeatedly crosses hybrid progeny, for example a first generation hybrid (F₁), back to one of the parents of the hybrid progeny. Backcrossing can be used to introduce one or more single locus conversions from one genetic background into another.

Commercial Soybean: A soybean seed or plant containing neither the Kunitz allele nor the SG-ULTI allele.

Crossing: The mating of two parent plants.

Cross-pollination: Fertilization by the union of two gametes from different plants.

Down-regulatory mutation: For the purposes of this application a down regulatory mutation is defined as a mutation that reduces the expression levels of a protein from a given gene. Thus a down-regulatory mutation comprises null mutations and reduced mutations.

F₁ Hybrid: The first generation progeny of the cross of two non-isogenic plants.

Genotype: The genetic constitution of a cell or organism.

INDEL: Genetic mutations resulting from insertion or deletion of nucleotide sequence.

Industrial use: A non-food and non-feed use for a soybean plant. The term “soybean plant” includes plant parts and derivatives of a soybean plant.

Kunitz allele: An allele of the Kunitz trypsin inhibitor gene, KTi3, containing nucleotides that differ from the wild-type gene at positions +481, +486, and +487, and result in a frameshift mutation causing the Kunitz phenotype as described in Orf and Hymowitz, J. Am. Oil Chem. Soc., 56:722-726 (1979) and Jofuku, et al., The Plant Cell, 1:427-435 (1989). The soybean variety, carrying only this Kunitz allele for reduced trypsin inhibitor activity, is referred to as the “Kunitz line.” These lines are readily available to the public.

Kunitz Phenotype: The trypsin inhibitor activity (in trypsin inhibitor units, TIU) found in soybeans carrying, as the only trypsin inhibitor gene mutation, the Kunitz allele, characterized as having at least a 30% reduction in trypsin inhibitor activity compared to commercial soybean lines having no mutations in trypsin inhibitor genes.

Linkage: A phenomenon wherein alleles on the same chromosome tend to segregate together more often than expected by chance if their transmission was independent.

Marker: A readily detectable phenotype, preferably inherited in codominant fashion (both alleles at a locus in a diploid heterozygote are readily detectable), with no environmental variance component, i.e., heritability of 1.

Non-transgenic mutation: A mutation that is naturally occurring or induced by conventional methods (e.g., exposure of plants to radiation or mutagenic compounds), not including mutations made using recombinant DNA techniques.

Null phenotype: A null phenotype as used herein means that a given protein is not expressed at levels that can be detected.

Phenotype: The detectable characteristics of a cell or organism, which characteristics are the manifestation of gene expression.

Reduced Trypsin Inhibitor Activity: As used herein “reduced trypsin inhibitor activity” means seed from plants comprising the SG-ULTI allele have reduced trypsin inhibitor activity, measured in an assay as trypsin inhibitor units (TIU), relative to plants with an identical genetic background that lack the mutation.

Reduced Levels of Trypsin Inhibitor Protein: As used herein “reduced levels of trypsin inhibitor protein” means seed from plants comprising the SG-ULTI allele have reduced trypsin inhibitor protein levels as compared to plants with an identical genetic background that lack the mutation.

SG-ULTI Allele: The novel, non-Kunitz allele in soybean line 435.TCS, causing a reduction in trypsin inhibitor activity, which is proposed to affect one or more of the Bowman-Birk trypsin inhibitor genes, other trypsin inhibitor genes, or sites affecting the expression or other regulation of trypsin inhibitor activity.

### BRIEF DESCRIPTION OF THE DRAWINGS

FIG. 1 illustrates a comparison of total trypsin inhibitor units (TIU) between proprietary line 435.TCS and the Kunitz line. Seed samples were produced in Maryland and the Queenstown research station in the summer of 2009. TIU units were measured by Eurofins Scientific Inc. on 10 gram seed samples. The proprietary 435.TCS variety does not carry a mutant allele of the Kunitz gene, but has mutant allele SG-ULTI and has TIU values lower than the Kunitz soybean line.

FIG. 2 shows a comparison of total trypsin inhibitor units (TIU) between a commercially available line (388.TC), Kunitz, and 435.TCS×Kunitz hybrid lines. Soybeans for the comparison were grown side-by-side in the same field. Soybean 388.TC has neither mutant trypsin inhibitor allele.

FIG. 3 displays a range of trypsin inhibitor units across two 2009 field experiments. Soybean samples were assayed for the presence or absence of the Kunitz trypsin inhibitor gene in the Schillinger Genetics molecular lab. Each point on the x-axis represents a single entry in the experiment and has been sorted by Kunitz trypsin inhibitor genotype as determined by the Schillinger Genetics molecular research lab. Seeds samples were produced in 2009 in Maryland (either Galena or Queenstown locations). The bars numbered 1-15 are seed samples segregating for the Kunitz trypsin inhibitor gene. The bars numbered 16-58 are seed samples that carry the Kunitz trypsin inhibitor mutation. Sample 47 is a seed sample from the uncrossed Kunitz line. The bars numbered 59-67 are seed samples that do not carry the Kunitz trypsin inhibitor mutation.

FIG. 4 shows the environmental effect on trypsin inhibitor units across three locations: Maryland-Galena (MDGA), Iowa-Lenox (IALX), and Iowa-Grinnell (IAGR). Each point on the x-axis represents a single entry in the experiment and has been sorted by Kunitz trypsin inhibitor genotype as determined by the Schillinger Genetics molecular research lab. Entries 1-7 are seed samples that are segregating for the Kunitz trypsin inhibitor gene. Entries 8-23 are seed samples carry the mutation in the Kunitz trypsin Inhibitor gene. Entries 24-27 are entries that do not carry a mutant allele of the Kunitz trypsin inhibitor gene.

### DETAILED DESCRIPTION OF THE INVENTION

The present invention includes plants and plant products derived from proprietary Kunitz soybean line having reduced trypsin inhibitor activity (measured as trypsin inhibitor units, TIU), crossed with the proprietary 435.TCS soybean line that has even lower TIU values. The 435.TCS soybean line has a wild-type Kunitz trypsin inhibitor gene, so the mutation that causes low trypsin inhibitor activity in this proprietary line must be located in another gene. This novel mutation has been designated SG-ULTI. The SG-ULTI mutant allele results from a mutation in one of the Bowman-Birk or other trypsin inhibitors that occur in soybeans. Alternatively, the disruption of genes involved in the modulation of the Bowman-Birk or other trypsin inhibitors are responsible for the observed phenotype. The resulting progeny from the 435.TCS×Kunitz cross have as low as 35% trypsin inhibitor activity of the Kunitz line, and as low as 15% of the commercial soybean varieties of soybean seeds.

In a preferred embodiment a soybean plant possesses the combined presence of the Kunitz allele for reduced trypsin inhibitor formation in the seeds along with the SG-ULTI mutant allele This combination is one aspect of the present invention and has been found to produce an unusually low expression for trypsin inhibitor in the resulting progeny seeds of a cross of a Kunitz allele into the 435.TCS background, thereby yielding a seed having as low as about 15% of the wild-type trypsin inhibitor activity. A seed or seed product is made possible in this instance that is particularly well suited for consumption without extensive processing to remove trypsin inhibitor. The present invention provides, in one embodiment, a plant of an agronomically elite soybean variety with reduced seed trypsin inhibitor content, comprising non-transgenic mutations which produce a reduced phenotype of trypsin inhibitor content and activity. A reduced seed trypsin inhibitor content was measured, for example, with respect to a plant of the same genotype but lacking the mutations. In specific embodiments, the non-transgenic mutations conferred a reduced seed trypsin inhibitor content. Thus, the plants of the current invention comprise, in one aspect, seeds with low trypsin inhibitor content. In certain embodiments, the seed trypsin inhibitor content for plants of the invention was about 15-35% or less of the TIU value of commercially available seeds.

In certain embodiments of the invention, soybean plants are provided that further comprise a mutation called the SG-ULTI allele, which confers even greater reduced levels of trypsin inhibitor protein. For example plants comprising a non-transgenic mutation that confers reduced trypsin inhibitor may have a trypsin inhibitor protein content of less than about 35% TIU value than commercial soybean varieties. In certain cases, the mutation conferring reduced trypsin inhibitor protein content was a non-transgenic mutation.

In some embodiments of the invention, the soybean plants have a reduced level of trypsin inhibitor activity. In some embodiments of the invention, a soybean plant or seed having the Kunitz allele and the SG-ULTI phenotype of reduced trypsin inhibitor activity has a range from about 8%, 10%, 15%, 18%, 20%, 25%, 30%, 40%, 43%, 50%, to about 60% and including all integers and fractions thereof of the trypsin inhibitor activity level of commercial soybean seeds.

In some embodiments of the invention, a soybean plant or seed having the Kunitz allele and the SG-ULTI phenotype of reduced trypsin inhibitor activity has from about 3,500 TIU to about 30,000 TIU, in comparison to a TIU of about 46,000 to 50,000 in commercial soybean lines. For example, a soybean plant or seed having the SG-ULTI phenotype of reduced trypsin inhibitor activity (in TIU) having a range from about 3,500, 4,000, 4,500, 4,800, 5,000, 5,400, 5,500, 6,000, 6,500, 7,000, 7,900, 8,000, 9,000, 10,000, 11,000, to 12,000 and including all integers and fractions thereof.

The presence of the SG-ULTI mutant allele can be tested using any suitable means, such as, for example, by obtaining a sample of the soybean plant or seed, and assaying the material for the presence of the mutant sequence by the use of general molecular techniques such as PCR, DNA hybridization using a nucleic acid probe, RFLP analysis, or nucleic acid sequencing methods.

Similarly, the presence of the Kunitz allele can be tested, for example, by obtaining a sample of the soybean plant or seed, and assaying the material for the presence of the mutant sequence by the use of general molecular techniques such as PCR, hybridization with a labeled nucleic acid probe, or nucleic acid sequencing methods.

The presence of the SG-ULTI mutant allele can also be detected by testing for the level of trypsin inhibitor activity in the plant material. For example, a seed or other sample of plant material can be obtained and assayed for trypsin inhibitor units (TIU) using methods known in the art. If the TIU level is lower than the TIU level of a plant having the Kunitz allele alone, the presence of both the Kunitz allele and the SG-ULTI mutant allele are determined.

Plant parts are also provided by the invention. Parts of a plant of the invention include, but are not limited to, pollen, ovules, meristems, cells, and seed. Cells of the invention may further comprise regenerable cells, such as embryos, meristematic cells, pollen, leaves, roots, root tips, and flowers. Thus, these cells could be used to regenerate plants of the invention.

Also provided herein are parts of the seeds of a plant according to the invention. Thus, crushed seed, and meal or flour made from seed according to the invention, is also provided as part of the invention. The invention further comprises a method for making soy meal or flour comprising crushing or grinding seed according to the invention. Such soy flour or meal according to the invention may comprise genomic material of the plant of the invention. In one embodiment, the food may be defined as comprising the genome of such a plant. In further embodiments soy meal or flour of the invention may be defined as comprising reduced trypsin inhibitor content, as compared to meal or flour made from seeds of a plant with an identical genetic background, but not comprising the non-transgenic, mutant alleles.

In yet a further aspect of the invention there is provided a method for producing a soybean seed, comprising crossing the plant of the invention with itself or with a second soybean plant. Thus, this method comprises preparing a hybrid soybean seed by crossing a plant of the invention with a second, distinct, soybean plant.

Still another aspect of the invention is a method of producing a food product for human or animal consumption comprising: (a) obtaining a plant of the invention; (b) cultivating the plant to maturity; and (c) preparing a food product from the plant. In certain embodiments of the invention, the food product may be protein concentrate, protein isolate, meal, oil, flour or soybean hulls. In some embodiments, the food product may comprise beverages such as soymilk and other nutritional beverages, infused foods, sauces, condiments, salad dressings, fruit juices, syrups, desserts, icings and fillings, soft frozen products, confections, or intermediate foods. Foods produced from the plants of the invention may comprise reduced trypsin inhibitor content and thus be of greater nutritional value than foods made with typical soybean varieties.

In further embodiments, a plant of the invention further comprises a transgene. For example, a plant may comprise transgenes conferring herbicide tolerance, disease resistance, insect and pest resistance, altered fatty acid, protein or carbohydrate metabolism, increased grain yield, altered plant maturity and/or altered morphological characteristics. For example, a herbicide tolerance transgene may comprise a glyphosate resistance gene.

In certain embodiments, a plant of the invention is defined as prepared by a method wherein a plant comprising non-transgenic mutations conferring a reduced trypsin inhibitor content is crossed with a plant comprising agronomically elite characteristics. The progeny of this cross may be assayed for agronomically elite characteristics and mutant trypsin inhibitor protein content, and progeny plants selected based on these characteristics, thereby generating the plant of the invention. Thus in certain embodiments, a plant of the invention was produced by crossing a selected starting variety with a second soybean plant comprising agronomically elite characteristics.

The current invention also provides a method of plant breeding wherein a plant is assayed for the presence of a polymorphism in a soybean plant genomic region associated with trypsin inhibitor and alleles, comprising selecting the plant and crossing the plant with a second soybean plant to produce progeny. In some embodiments, the method of the invention comprise selecting a progeny plant by assaying the plant for a polymorphism associated with a reduced trypsin inhibitor or related phenotype and crossing the plant with a second soybean plant to produce further progeny plants. In certain embodiments of the invention, the second soybean plant comprises agronomically elite characteristics. The method of the invention also further comprises selecting a soybean plant comprising the polymorphism and agronomically elite characteristics. Thus, the invention enables the introduction of non-transgenic mutations conferring a trypsin inhibitor mutant phenotype and reduced seed trypsin inhibitor content into agronomically elite soybean plants. A method of the present invention may be repeated 1, 2, 3, 4, 5, 10, 15, 20, or more times as desired to select agronomically elite progeny with polymorphisms indicative of non-transgenic mutations at trypsin inhibitor and/or other alleles at each step. In a further embodiment, a method of the invention may further comprise selecting a plant comprising polymorphisms indicative of a non-transgenic mutation in trypsin inhibitor and other alleles.

In some embodiments of the current invention, non-transgenic mutations conferring a reduced trypsin inhibitor phenotype may comprise mutations in Kunitz alleles. In certain embodiments, the mutant Kunitz alleles are detected using genetic markers comprising polymorphisms within the Kunitz allele. In further aspects of the invention, plants with a reduced trypsin inhibitor phenotype comprise another mutant allele. In some cases, the mutant allele comprises a deletion, such as a deletion of the promoter region of the gene. In other embodiments, mutant alleles can be detected using molecular markers. In certain aspects of the invention, mutant alleles may be detected with markers that are closely linked. Thus, in other aspects of the invention, mutant alleles may be detected with closely linked markers. In further embodiments, mutant alleles comprise point mutations such as a SNP that reduces or abrogates the translation of protein. Single nucleotide polymorphism (SNP) markers may be detected, for example using fluorescently labeled oligonucleotides.

In some aspects of the current invention, non-transgenic mutations conferring a reduced trypsin inhibitor phenotype may comprise mutations in alleles of genes other than Kunitz genes, such as genes for the Bowman-Birk trypsin inhibitors. In certain embodiments, the mutant SG-ULTI allele is detected using genetic markers comprising polymorphisms within the 50 cM of an SG-ULTI mutant allele. In further aspects of the invention, plants with reduced trypsin inhibitor phenotype comprise another mutant allele. In some cases, the mutant allele comprises a deletion, such as a deletion of the promoter region of the gene. In other embodiments, mutant alleles can be detected using molecular markers. In certain aspects of the invention, mutant alleles may be detected with markers that are closely linked. Thus, in other aspects of the invention, mutant alleles may be detected with closely linked markers. In further embodiments, mutant alleles comprise point mutations such as a SNP that reduces or abrogates the translation of protein. SNP markers may be detected, for example using fluorescently labeled oligonucleotides.

In some embodiments, a method of the invention further comprises selecting plants with markers indicative of an SG-ULTI allele. Thus, methods of marker-assisted plant breeding according to the invention may be used to produce soybeans that have reduced or undetectable trypsin inhibitor content.

In certain embodiments of the invention, mutations conferring a Kunitz phenotype may comprise mutations in a gene encoding the Kunitz allele. In a particular embodiment, mutations conferring a Kunitz phenotype comprise mutations in the KTI3 gene, also termed “KTIA” (Kim et al., Theor. Appl. Genet., 121(4):751-60 (2010); Genbank Accession No. S45092). In one embodiment of the invention, the mutant alleles conferring a Kunitz phenotype are detected using genetic markers comprising polymorphisms within the Kunitz allele. In certain embodiments, Kunitz alleles are detected using one or more INDELs or SNPs located within the KTI3 gene. Such selection may thus be based on marker information (plant genotype) rather than on enzymatic analysis of trypsin activity or analysis of Kunitz trypsin inhibitor content.

Embodiments discussed in the context of a method and/or composition of the invention may be employed with respect to any other method or composition described herein. Thus, an embodiment pertaining to one method or composition may be applied to other methods and compositions of the invention as well.

### EXAMPLES

The ultra-low trypsin inhibitor phenotype was developed by crossing 435.TCS and the Kunitz soybean line in 2006. Following this cross, lines were advanced to F₃ using the modified pod pick method. Single F₅ and F₆ plants were selected based on basic agronomics (plant type: stature and structure). Selected F₇ lines were analyzed for total trypsin inhibitor content and the novel phenotype was discovered. The ultra-low trypsin inhibitor phenotype was not found in segregants from other genetic crosses.

Without being bound by any specific mechanism, it appears that the novel ultra-low trypsin inhibitor phenotype is the result of the combination of the Kunitz allele with the novel mutation or mutations giving rise to the previously unobserved SG-ULTI phenotype. This latter novel mutation has been designated as SG-ULTI by Schillinger Genetics.

### Example 1

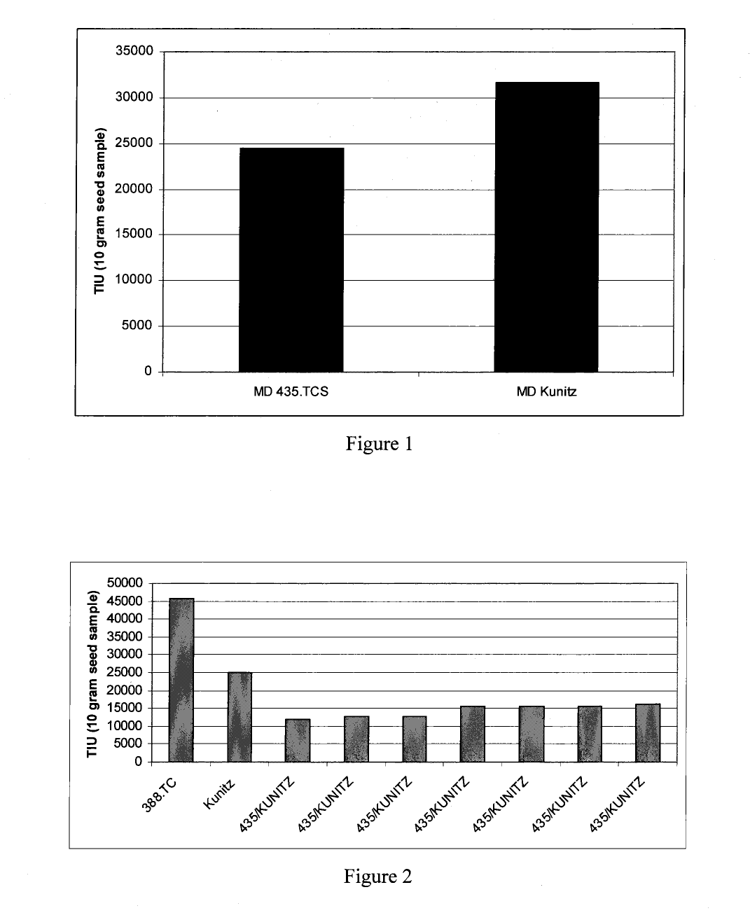

In the present application, newly derived soybeans having an ultra-low trypsin inhibitor content and activity that is lower than previously known Kunitz lines of soybeans have been developed. Trypsin inhibition activity is expressed in trypsin inhibitor units (TIU). These new ultra-low trypsin inhibitor soybeans resulted from crosses of proprietary 435.TCS soybean line with the Kunitz soybean line. The 435.TCS line was known from molecular testing to have a wild-type copy of the Kunitz allele. Thus, the observed reduction in trypsin inhibitor activity was due to a mutation at another locus, perhaps within one or more genes of the Bowman-Birk class of trypsin inhibitors. This separate allele is termed the SG-ULTI allele of the present invention. In 2009, the 435.TCS soybean line was approximately 23% lower trypsin inhibitor activity (FIG. 1) than the Kunitz line when grown side-by-side under identical conditions. This result was unexpected because the Kunitz variety has previously been known as having the lowest values for trypsin inhibitor activity. In 2010, Kunitz exhibited lower trypsin inhibitor activity than line 435.TCS (Table 1). In view of these results, getting significantly reduced trypsin inhibitor content and activity in progeny of Kunitz×435.TCS was unexpected. The ultra-low trypsin inhibitor content and activity of 435.TCS/Kunitz hybrids compared with the parental lines and commercial lines is the result of complex multi-gene interactions.

### Example 2

In 2009 we compared the Kunitz line to a commercial soybean line known as 388.TC and to several novel soybean lines produced by crossing the 435.TCS soybean line with the Kunitz soybean line (FIG. 2). The Kunitz line, carrying only the Kunitz mutation to reduce trypsin inhibitor activity, by itself had a reduction in activity of at least 45% compared to the commercial line, which carried neither the Kunitz allele, nor the SG-ULTI phenotype. When the Kunitz line was compared to the Kunitz×435.TCS hybrid progeny, these new progeny lines showed a further reduction in trypsin inhibitor activity of from about 36% to about 52%, when compared to the Kunitz line. These reductions in trypsin inhibitor activity in the Kunitz×435.TCS hybrid lines correspond to an overall reduction in trypsin inhibitor activity, compared to commercial soybean varieties, of about 65 to 74% in these samples. These results demonstrate the ultra-low trypsin inhibitor phenotype characteristic of the Kunitz×435.TCS hybrids.

### Example 3

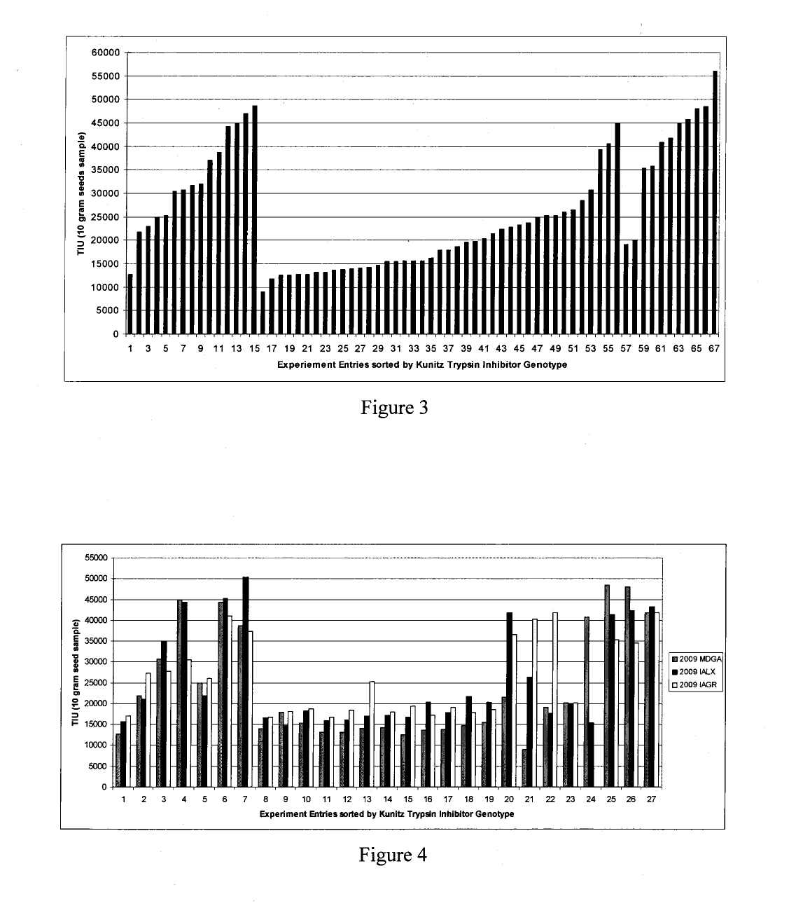

An expanded experiment was conducted to compare several novel hybrid soybean lines to lines heterozygous for the Kunitz allele, to a line homozygous for the Kunitz allele, and to several lines that were wild type for the Kunitz allele. FIG. 3 demonstrates that while there is variability within each class of soybean line, clear differences between each class were observed.

These experiments were performed at two different sites in Maryland (one in Galena and another in Queenstown). While the Kunitz soybean line (Sample 47, FIG. 3) had a TIU of 25,000, the Kunitz×435.TCS hybrids achieved TIU values as low as 9,000. Even when the Kunitz gene is not mutated, 435.TCS samples reach TIU levels around 20,000 (data points 57 and 58). Many of the data points in this graph are from soybean lines which share similar pedigrees of Kunitz combined with the Schillinger Genetics variety 435.TCS (see FIG. 3).

### Example 4

The reduction in total trypsin inhibitor values for the progeny of a 435.TCS×Kunitz cross is detectable across environments, but there was some environmental effect on the phenotype because a range of values exist. There may be additional genes controlling the phenotype each sub-line may have a unique combination of homozygous and heterozygous alleles of the Bowman-Birk type or other trypsin inhibitors. Data points from an experiment conducted in three US locations (Maryland-Galena, Iowa-Lenox, and Iowa-Grinnell) are shown in FIG. 4. These data points were sorted by their Kunitz genotype as determined in the Schillinger Genetics molecular lab. Generally, it was possible to tell by a single data point among the 435.TCS×Kunitz hybrids if a line has a reduction in TIU relative to either plants wild-type for the Kunitz gene or heterozygous plants, but an individual soybean line may have a range of TIU values when compared over the different growing environments. While samples from plants either heterozygous for or lacking the Kunitz mutant allele show notable variability both within and between individuals, and generally higher TIU values, the 435.TCS×Kunitz hybrids represented by samples 8-23 in FIG. 4 show a marked overall reduction in trypsin inhibitor activity, with most samples having limited variability across environments.

### Example 5

In 2010 we analyzed the trypsin inhibitor values for 435.TCS, Kunitz, several commercial varieties, and novel lines 029K417, 029K418, 037K421, and 031K420, produced by crossing the 435.TCS soybean line with the Kunitz soybean line (Table 1). The Kunitz and 435.TCS lines were grown in Illinois, the commercial varieties were grown in southern Illinois, and the new hybrid lines were grown in Iowa. As shown in Table 1, column 2, hybrid sub-lines derived from 435.TCS Kunitz progeny exhibited a reduction in trypsin inhibitor values (column 3). The highest and lowest values obtained for each line are in bold. These results demonstrate the unexpected ultra-low trypsin inhibitor phenotype characteristic of the Kunitz×435.TCS hybrids.

TABLE 1

| | Variety | TIU |
|---|---|---|
| Parent line | 435.TCS | 38000 |
| Parent line | Kunitz | 29000 |
| Commercial lines | 348.TCS | 49000 |
| | P93B82 | 50300 |
| | XC4510 | 46900 |
| Novel sub-lines of | 029K417-1 | 7100 |
| 029K417 | 029K417-2 | 6000 |
| | 029K417-3 | 6200 |
| | 029K417-4 | 8600 |
| | 029K417-5 | 4900 |
| | 029K417-6 | 5900 |
| | 029K417-7 | 5900 |
| | 029K417-8 | 5400 |
| | 029K417-9 | 4800 |
| | 029K417-10 | 5500 |
| | 029K417-11 | 4900 |
| Novel sub-lines of | 029K418-1 | 7900 |
| 029K418 | 029K418-2 | 7500 |
| Novel sub-lines of | 037K421-1 | 9500 |
| 037K421 | 037K421-2 | 6700 |
| | 037K421-3 | 7900 |
| | 037K421-4 | 8500 |
| | 037K421-5 | 9300 |
| | 037K421-6 | 7000 |
| | 037K421-7 | 6700 |
| | 037K421-8 | 7100 |
| | 037K421-9 | 8500 |
| | 037K421-10 | 11200 |
| | 037K421-11 | 7300 |
| | 037K421-12 | 7700 |
| | 037K421-13 | 7900 |
| | 037K421-14 | 9000 |
| Novel sub-lines of | 031K420-1 | 6700 |
| 031K420 | 031K420-2 | 7900 |
| | 031K420-3 | 7400 |
| | 031K420-4 | 9500 |
| | 031K420-5 | 6900 |
| | 031K420-6 | 7100 |

The advent of new molecular biological techniques has allowed the isolation and characterization of genetic elements with specific functions, such as encoding specific protein products. Scientists in the field of plant biology developed a strong interest in engineering the genome of plants to contain and express foreign genetic elements, or additional, or modified versions of native or endogenous genetic elements in order to alter the traits of a plant in a specific manner. Any DNA sequences, whether from a different species or from the same species, which are introduced into the genome using transformation or various breeding methods are referred to herein collectively as “transgenes.” In some embodiments of the invention, a transgenic variant of the ultra-low trypsin inhibitor soybean of the present invention may contain at least one transgene but could contain at least 1, 2, 3, 4, 5, 6, 7, 8, 9, 10, and/or no more than 15, 14, 13, 12, 11, 10, 9, 8, 7, 6, 5, 4, 3, or 2. Over the last fifteen to twenty years several methods for producing transgenic plants have been developed, and the present invention also relates to transgenic variants of the claimed ultra-low trypsin inhibitor soybean.

### FURTHER EMBODIMENTS OF THE INVENTION

Nucleic acids or polynucleotides refer to RNA or DNA that is linear or branched, single or double stranded, or a hybrid thereof. The term also encompasses RNA/DNA hybrids. These terms also encompass untranslated sequence located at both the 3′ and 5′ ends of the coding region of the gene: at least about 1000 nucleotides of sequence upstream from the 5′ end of the coding region and at least about 200 nucleotides of sequence downstream from the 3′ end of the coding region of the gene. Less common bases, such as inosine, 5-methylcytosine, 6-methyladenine, hypoxanthine and others can also be used for antisense, dsRNA and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. The antisense polynucleotides and ribozymes can consist entirely of ribonucleotides, or can contain mixed ribonucleotides and deoxyribonucleotides. The polynucleotides of the invention may be produced by any means, including genomic preparations, cDNA preparations, in vitro synthesis, RT-PCR, and in vitro or in vivo transcription.

Primers are isolated nucleic acids that are annealed to a complimentary target DNA strand by nucleic acid hybridization to form a hybrid between the primer and the target DNA strand, then extended along the target DNA strand by a polymerase, such as DNA polymerase. Primer pairs or sets can be used for amplification of a nucleic acid molecule, for example, by the polymerase chain reaction (PCR) or other conventional nucleic-acid amplification methods.

A “probe” is an isolated nucleic acid to which is attached a conventional detectable label or reporter molecule, such as a radioactive isotope, ligand, chemiluminescent agent, or enzyme. Such a probe is complimentary to a strand of a target nucleic acid. Probes according to the present invention include not only deoxyribonucleic or ribonucleic acids but also polyamides and other probe materials that bind specifically to a target DNA sequence and can be used to detect the presence of that target DNA sequence.

Primers and probes are generally between 10 and 15 nucleotides or more in length, primers and probes can also be at least 20 nucleotides or more in length, or at least 25 nucleotides or more, or at least 30 nucleotides or more in length. Such primers and probes hybridize specifically to a target sequence under high stringency hybridization conditions. Primers and probes according to the present invention may have complete sequence complementary with the target sequence, although probes differing from the target sequence and which retain the ability to hybridize to target sequences may be designed by conventional methods.

Stringent conditions or stringent bybridization conditions refer to conditions under which a probe will hybridize to its target sequence, to a detectably greater degree than to other sequences. Stringent conditions are target-sequence-dependent and will differ depending on the structure of the polynucleotide. By controlling the stringency of the hybridization and/or wash conditions, target sequences can be identified which are 100% complementary to the probe (homologous probing). Alternatively, stringency conditions can be adjusted to allow some mismatching in sequences so that lower degrees of similarity are detected. Longer sequences hybridize specifically at higher temperatures. An extensive guide to the hybridization of nucleic acids is found in Current Protocols in Molecular Biology, Chapter 2, Ausubel et al., Eds., Greene Publishing and Wiley-Interscience: New York (1995), and also Sambrook et al. (2001) Molecular Cloning: A Laboratory Manual (5th Ed. Cols Spring Harbor Laboratory, Cold Spring Harbor, N.Y.).

Numerous methods for plant transformation have been developed, including biological and physical plant transformation protocols. See, for example, Miki et al., “Procedures for Introducing Foreign DNA into Plants,” in Methods in Plant Molecular Biology and Biotechnology, Glick and Thompson Eds., CRC Press, Inc., Boca Raton, pp. 67-88 (1993), and Armstrong, “The First Decade of Maize Transformation: A Review and Future Perspective,” Maydica, 44:101-109 (1999). In addition, expression vectors and in vitro culture methods for plant cell or tissue transformation and regeneration of plants are available. See, for example, Gruber, et al., “Vectors for Plant Transformation,” in Methods in Plant Molecular Biology and Biotechnology, Glick and Thompson Eds., CRC Press, Inc., Boca Raton, pp. 89-119 (1993).

A genetic trait which has been engineered into the genome of a particular soybean plant may then be moved into the genome of another variety using traditional breeding techniques that are well known in the plant breeding arts. For example, a backcrossing approach is commonly used to move a transgene from a transformed soybean variety into an already developed soybean variety, and the resulting backcross conversion plant would then comprise the transgene(s).

Various genetic elements can be introduced into the plant genome using transformation. These elements include, but are not limited to, genes, coding sequences, inducible, constitutive and tissue specific promoters, enhancing sequences, and signal and targeting sequences. For example, see the traits, genes, and transformation methods listed in U.S. Pat. No. 6,118,055.

Plant transformation involves the construction of an expression vector which will function in plant cells. Such a vector comprises DNA comprising a gene under the control of, or operatively linked to, a regulatory element (for example, a promoter). The expression vector may contain one or more such operably linked gene/regulatory element combinations. The vector(s) may be in the form of a plasmid and can be used alone or in combination with other plasmids to provide transformed soybean plants using transformation methods as described below to incorporate transgenes into the genetic material of the soybean plant(s).

### Expression Vectors for Soybean Transformation: Marker Genes

Expression vectors include at least one genetic marker operably linked to a regulatory element (for example, a promoter) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or an herbicide, or genes that encode an altered target which is insensitive to the inhibitor. A few positive selection methods are also known in the art.

One commonly used selectable marker gene for plant transformation is the neomycin phosphotransferase II (nptII) gene which, when under the control of plant regulatory signals, confers resistance to kanamycin (Fraley, et al., Proc. Natl. Acad. Sci. USA, 80:4803 (1983)). Another commonly used selectable marker gene is the hygromycin phosphotransferase gene which confers resistance to the antibiotic hygromycin (Vanden Elzen, et al., Plant Mol. Biol., 5:299 (1985)).

Additional selectable marker genes of bacterial origin that confer resistance to antibiotics include gentamycin acetyl transferase, streptomycin phosphotransferase and aminoglycoside-3′-adenyl transferase, the bleomycin resistance determinant (Hayford, et al., Plant Physiol., 86:1216 (1988); Jones, et al., Mol. Gen. Genet., 210:86 (1987); Svab, et al., Plant Mol. Biol., 14:197 (1990); Hille, et al., Plant Mol. Biol., 7:171 (1986)). Other selectable marker genes confer resistance to herbicides such as glyphosate, glufosinate, or bromoxynil (Comai, et al., Nature, 317:741-744 (1985); Gordon-Kamm, et al., Plant Cell, 2:603-618 (1990); Stalker, et al., Science, 242:419-423 (1988)).

Selectable marker genes for plant transformation not of bacterial origin include, for example, mouse dihydrofolate reductase, plant 5-enolpyruvylshikimate-3-phosphate synthase, and plant acetolactate synthase (Eichholtz, et al., Somatic Cell Mol. Genet., 13:67 (1987); Shah, et al., Science, 233:478 (1986); Charest, et al., Plant Cell Rep., 8:643 (1990)).

Another class of marker genes for plant transformation requires screening of presumptively transformed plant cells, rather than direct genetic selection of transformed cells, for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used genes for screening presumptively transformed cells include β-glucuronidase (GUS), β-galactosidase, luciferase, and chloramphenicol acetyltransferase (Jefferson, R. A., Plant Mol. Biol. Rep., 5:387 (1987); Teeri, et al., EMBO J., 8:343 (1989); Koncz, et al., Proc. Natl. Acad. Sci. USA, 84:131 (1987); DeBlock, et al., EMBO J., 3:1681 (1984)).

In vivo methods for visualizing GUS activity that do not require destruction of plant tissue are available (Molecular Probes, Publication 2908, IMAGENE GREEN, pp. 1-4 (1993); Naleway, et al., J. Cell Biol., 115:151a (1991)). However, these in vivo methods for visualizing GUS activity have not proven useful for recovery of transformed cells because of low sensitivity, high fluorescent backgrounds, and limitations associated with the use of luciferase genes as selectable markers.

More recently, a gene encoding Green Fluorescent Protein (GFP) has been utilized as a marker for gene expression in prokaryotic and eukaryotic cells (Chalfie, et al., Science, 263:802 (1994)). GFP and mutants of GFP may be used as screenable markers.

### Expression Vectors for Soybean Transformation: Promoters

Genes included in expression vectors must be driven by a nucleotide sequence comprising a regulatory element (for example, a promoter). Several types of promoters are well known in the transformation arts as are other regulatory elements that can be used alone or in combination with promoters.

As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred.” Promoters that initiate transcription only in a certain tissue are referred to as “tissue-specific.” A “cell-type” specific promoter primarily drives expression in certain cell types in one or more organs, for example, vascular cells in roots or leaves. An “inducible” promoter is a promoter which is under environmental control. Examples of environmental conditions that may effect transcription by inducible promoters include anaerobic conditions or the presence of light. Tissue-specific, tissue-preferred, cell-type specific, and inducible promoters constitute the class of “non-constitutive” promoters. A “constitutive” promoter is a promoter that is active under most environmental conditions.

A. Inducible Promoters—An inducible promoter is operably linked to a gene for expression in soybean. Optionally, the inducible promoter is operably linked to a nucleotide sequence encoding a signal sequence which is operably linked to a gene for expression in soybean. With an inducible promoter the rate of transcription increases in response to an inducing agent.

Any inducible promoter can be used in the instant invention. See, Ward, et al., Plant Mol. Biol., 22:361-366 (1993). Exemplary inducible promoters include, but are not limited to, that from the ACEI system which responds to copper (Mett, et al., Proc. Natl. Acad. Sci. USA, 90:4567-4571 (1993)); In2 gene from maize which responds to benzenesulfonamide herbicide safeners (Hershey, et al., Mol. Gen Genetics, 227:229-237 (1991); Gatz, et al., Mol. Gen. Genetics, 243:32-38 (1994)); or Tet repressor from Tn10 (Gatz, et al., Mol. Gen. Genetics, 227:229-237 (1991)). A particularly preferred inducible promoter is a promoter that responds to an inducing agent to which plants do not normally respond. An exemplary inducible promoter is the inducible promoter from a steroid hormone gene, the transcriptional activity of which is induced by a glucocorticosteroid hormone (Schena, et al., Proc. Natl. Acad. Sci. USA, 88:0421 (1991)).

B. Constitutive Promoters—A constitutive promoter is operably linked to a gene for expression in soybean or the constitutive promoter is operably linked to a nucleotide sequence encoding a signal sequence which is operably linked to a gene for expression in soybean.

Many different constitutive promoters can be utilized in the instant invention. Exemplary constitutive promoters include, but are not limited to, the promoters from plant viruses such as the 35S promoter from CaMV (Odell, et al., Nature, 313:810-812 (1985)) and the promoters from such genes as rice actin (McElroy, et al., Plant Cell, 2: 163-171 (1990)); ubiquitin (Christensen, et al., Plant Mol. Biol., 12:619-632 (1989); Christensen, et al., Plant Mol. Biol., 18:675-689 (1992)); pEMU (Last, et al., Theor. Appl. Genet., 81:581-588 (1991)); MAS (Velten, et al., EMBO J., 3:2723-2730 (1984)); and maize H3 histone (Lepetit, et al., Mol. Gen. Genetics, 231:276-285 (1992); Atanassova, et al., Plant Journal, 2 (3): 291-300 (1992)). The ALS promoter, Xba1/Nco1 fragment 5′ to the Brassica napus ALS3 structural gene (or a nucleotide sequence similarity to said Xba1/Nco1 fragment), represents a particularly useful constitutive promoter. See, PCT Application WO 96/30530.

C. Tissue-Specific or Tissue-Preferred Promoters—A tissue-specific promoter is operably linked to a gene for expression in soybean. Optionally, the tissue-specific promoter is operably linked to a nucleotide sequence encoding a signal sequence which is operably linked to a gene for expression in soybean. Plants transformed with a gene of interest operably linked to a tissue-specific promoter produce the protein product of the transgene exclusively, or preferentially, in a specific tissue.

Any tissue-specific or tissue-preferred promoter can be utilized in the instant invention. Exemplary tissue-specific or tissue-preferred promoters include, but are not limited to, a root-preferred promoter such as that from the phaseolin gene (Murai, et al., Science, 23:476-482 (1983); Sengupta-Gopalan, et al., Proc. Natl. Acad. Sci. USA, 82:3320-3324 (1985)); a leaf-specific and light-induced promoter such as that from cab or rubisco (Simpson, et al., EMBO J., 4(11):2723-2729 (1985); Timko, et al., Nature, 318:579-582 (1985)); an anther-specific promoter such as that from LAT52 (Twell, et al., Mol. Gen. Genetics, 217:240-245 (1989)); a pollen-specific promoter such as that from Zm13 (Guerrero, et al., Mol. Gen. Genetics, 244:161-168 (1993)); or a microspore-preferred promoter such as that from apg (Twell, et al., Sex. Plant Reprod., 6:217-224 (1993)).

### Signal Sequences for Targeting Proteins to Subcellular Compartments

Transport of a protein produced by transgenes to a subcellular compartment, such as the chloroplast, vacuole, peroxisome, glyoxysome, cell wall, or mitochondrion, or for secretion into the apoplast, is accomplished by means of operably linking the nucleotide sequence encoding a signal sequence to the 5′ and/or 3′ region of a gene encoding the protein of interest. Targeting sequences at the 5′ and/or 3′ end of the structural gene may determine during protein synthesis and processing where the encoded protein is ultimately compartmentalized.

The presence of a signal sequence directs a polypeptide to either an intracellular organelle or subcellular compartment or for secretion to the apoplast. Many signal sequences are known in the art. See, for example, Becker, et al., Plant Mol. Biol., 20:49 (1992); Knox, C., et al., Plant Mol. Biol., 9:3-17 (1987); Lerner, et al., Plant Physiol., 91:124-129 (1989); Frontes, et al., Plant Cell, 3:483-496 (1991); Matsuoka, et al., Proc. Natl. Acad. Sci., 88:834 (1991); Gould, et al., J. Cell. Biol., 108:1657 (1989); Creissen, et al., Plant J., 2:129 (1991); Kalderon, et al., Cell, 39:499-509 (1984); Steifel, et al., Plant Cell, 2:785-793 (1990).

### Foreign Protein Genes and Agronomic Genes

With transgenic plants according to the present invention, a foreign protein can be produced in commercial quantities. Thus, techniques for the selection and propagation of transformed plants, which are well understood in the art, yield a plurality of transgenic plants which are harvested in a conventional manner, and a foreign protein can then be extracted from a tissue of interest or from total biomass. Protein extraction from plant biomass can be accomplished by known methods which are discussed, for example, by Heney and Orr, Anal. Biochem., 114:92-6 (1981).

According to a preferred embodiment, the transgenic plant provided for commercial production of foreign protein is a soybean plant. In another preferred embodiment, the biomass of interest is seed. For the relatively small number of transgenic plants that show higher levels of expression, a genetic map can be generated, primarily via conventional RFLP, PCR, and SSR analysis, which identifies the approximate chromosomal location of the integrated DNA molecule. For exemplary methodologies in this regard, see, Glick and Thompson, Methods in Plant Molecular Biology and Biotechnology, CRC Press, Inc., Boca Raton, 269:284 (1993). Map information concerning chromosomal location is useful for proprietary protection of a subject transgenic plant.

Wang, et al. discuss “Large Scale Identification, Mapping and Genotyping of Single-Nucleotide Polymorphisms in the Human Genome,” Science, 280:1077-1082 (1998), and similar capabilities are becoming increasingly available for the soybean genome. Map information concerning chromosomal location is useful for proprietary protection of a subject transgenic plant. If unauthorized propagation is undertaken and crosses made with other germplasm, the map of the integration region can be compared to similar maps for suspect plants to determine if the latter have a common parentage with the subject plant. Map comparisons would involve hybridizations, RFLP, PCR, SSR, and sequencing, all of which are conventional techniques. SNPs may also be used alone or in combination with other techniques.

Likewise, by means of the present invention, plants can be genetically engineered to express various phenotypes of agronomic interest. Through the transformation of soybean, the expression of genes can be altered to enhance disease resistance, insect resistance, herbicide resistance, agronomic, grain quality, and other traits. Transformation can also be used to insert DNA sequences which control or help control male-sterility. DNA sequences native to soybean, as well as non-native DNA sequences, can be transformed into soybean and used to alter levels of native or non-native proteins. Various promoters, targeting sequences, enhancing sequences, and other DNA sequences can be inserted into the genome for the purpose of altering the expression of proteins. Reduction of the activity of specific genes (also known as gene silencing or gene suppression) is desirable for several aspects of genetic engineering in plants.

Many techniques for gene silencing are well known to one of skill in the art, including, but not limited to, knock-outs (such as by insertion of a transposable element such as mu (Vicki Chandler, The Maize Handbook, Ch. 118 (Springer-Verlag 1994)) or other genetic elements such as a FRT, Lox, or other site specific integration site, antisense technology (see, e.g., Sheehy, et al., PNAS USA, 85:8805-8809 (1988); and U.S. Pat. Nos. 5,107,065, 5,453,566, and 5,759,829); co-suppression (e.g., Taylor, Plant Cell, 9:1245 (1997); Jorgensen, Trends Biotech., 8(12):340-344 (1990); Flavell, PNAS USA, 91:3490-3496 (1994); Finnegan, et al., Bio/Technology, 12:883-888 (1994); Neuhuber, et al., Mol. Gen. Genet., 244:230-241 (1994)); RNA interference (Napoli, et al., Plant Cell, 2:279-289 (1990); U.S. Pat. No. 5,034,323; Sharp, Genes Dev., 13:139-141 (1999); Zamore, et al., Cell, 101:25-33 (2000); Montgomery, et al., PNAS USA, 95:15502-15507 (1998)), virus-induced gene silencing (Burton, et al., Plant Cell, 12:691-705 (2000); Baulcombe, Curr. Op. Plant Bio., 2:109-113 (1999)); target-RNA-specific ribozymes (Haseloff, et al., Nature, 334: 585-591 (1988)); hairpin structures (Smith, et al., Nature, 407:319-320 (2000); WO 99/53050; WO 98/53083); MicroRNA (Aukerman & Sakai, Plant Cell, 15:2730-2741 (2003)); ribozymes (Steinecke, et al., EMBO J., 11:1525 (1992); Perriman, et al., Antisense Res. Dev., 3:253 (1993)); oligonucleotide mediated targeted modification (e.g., WO 03/076574 and WO 99/25853); Zn-finger targeted molecules (e.g., WO 01/52620, WO 03/048345, and WO 00/42219); and other methods or combinations of the above methods known to those of skill in the art.

Likewise, by means of the present invention, agronomic genes can be expressed in transformed plants. More particularly, plants can be genetically engineered to express various phenotypes of agronomic interest. Exemplary genes implicated in this regard include, but are not limited to, those categorized below:

### 1. Genes That Confer Resistance to Pests or Disease and That Encode:

A. Plant disease resistance genes. Plant defenses are often activated by specific interaction between the product of a disease resistance gene (R) in the plant and the product of a corresponding avirulence (Avr) gene in the pathogen. A plant variety can be transformed with one or more cloned resistance genes to engineer plants that are resistant to specific pathogen strains. See, for example, Jones, et al., Science, 266:789 (1994) (cloning of the tomato Cf-9 gene for resistance to Cladosporium fulvum); Martin, et al., Science, 262:1432 (1993) (tomato Pto gene for resistance to Pseudomonas syringae pv. tomato encodes a protein kinase); Mindrinos, et al., Cell, 78:1089 (1994) (Arabidopsis RSP2 gene for resistance to Pseudomonas syringae); McDowell & Woffenden, Trends Biotechnol., 21(4):178-83 (2003); and Toyoda, et al., Transgenic Res., 11 (6):567-82 (2002).

B. A gene conferring resistance to a pest, such as soybean cyst nematode. See, e.g., PCT Application WO 96/30517 and PCT Application WO 93/19181.

C. A Bacillus thuringiensis protein, a derivative thereof or a synthetic polypeptide modeled thereon. See, for example, Geiser, et al., Gene, 48:109 (1986), who disclose the cloning and nucleotide sequence of a Bt δ-endotoxin gene. Moreover, DNA molecules encoding δ-endotoxin genes can be purchased from American Type Culture Collection, Manassas, Va., for example, under ATCC Accession Nos. 40098, 67136, 31995, and 31998.

D. A lectin. See, for example, Van Damme, et al., Plant Molec. Biol., 24:25 (1994), who disclose the nucleotide sequences of several Clivia miniata mannose-binding lectin genes.

E. A vitamin-binding protein such as avidin. See, PCT Application US 93/06487, which teaches the use of avidin and avidin homologues as larvicides against insect pests.

F. An enzyme inhibitor, for example, a protease or proteinase inhibitor or an amylase inhibitor. See, for example, Abe, et al., J. Biol. Chem., 262:16793 (1987) (nucleotide sequence of rice cysteine proteinase inhibitor); Huub, et al., Plant Molec. Biol., 21:985 (1993) (nucleotide sequence of cDNA encoding tobacco proteinase inhibitor I); Sumitani, et al., Biosci. Biotech. Biochem., 57:1243 (1993) (nucleotide sequence of Streptomyces nitrosporeus α-amylase inhibitor); and U.S. Pat. No. 5,494,813 (Hepher and Atkinson, issued Feb. 27, 1996).

G. An insect-specific hormone or pheromone, such as an ecdysteroid or juvenile hormone, a variant thereof, a mimetic based thereon, or an antagonist or agonist thereof. See, for example, the disclosure by Hammock, et al., Nature, 344:458 (1990), of baculovirus expression of cloned juvenile hormone esterase, an inactivator of juvenile hormone.

H. An insect-specific peptide or neuropeptide which, upon expression, disrupts the physiology of the affected pest. For example, see the disclosures of Regan, J. Biol. Chem., 269:9 (1994) (expression cloning yields DNA coding for insect diuretic hormone receptor); Pratt, et al., Biochem. Biophys. Res. Comm., 163:1243 (1989) (an allostatin is identified in Diploptera puntata); Chattopadhyay, et al., Critical Reviews in Microbiology, 30(1):33-54 (2004); Zjawiony, J Nat Prod, 67(2):300-310 (2004); Carlini & Grossi-de-Sa, Toxicon, 40(11):1515-1539 (2002); Ussuf, et al., Curr Sci., 80(7):847-853 (2001); Vasconcelos & Oliveira, Toxicon, 44(4):385-403 (2004). See also, U.S. Pat. No. 5,266,317 to Tomalski, et al., which discloses genes encoding insect-specific, paralytic neurotoxins.

I. An insect-specific venom produced in nature by a snake, a wasp, etc. For example, see, Pang, et al., Gene, 116:165 (1992), for disclosure of heterologous expression in plants of a gene coding for a scorpion insectotoxic peptide.

J. An enzyme responsible for a hyperaccumulation of a monoterpene, a sesquiterpene, a steroid, hydroxamic acid, a phenylpropanoid derivative, or another non-protein molecule with insecticidal activity.

K. An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase, and a glucanase, whether natural or synthetic. See, PCT Application WO 93/02197 (Scott, et al.), which discloses the nucleotide sequence of a callase gene. DNA molecules which contain chitinase-encoding sequences can be obtained, for example, from the ATCC under Accession Nos. 39637 and 67152. See also, Kramer, et al., Insect Biochem. Molec. Biol., 23:691 (1993), who teach the nucleotide sequence of a cDNA encoding tobacco hornworm chitinase, and Kawalleck, et al., Plant Molec. Biol., 21:673 (1993), who provide the nucleotide sequence of the parsley ubi4-2 polyubiquitin gene, U.S. Pat. Nos. 7,145,060, 7,087,810, and 6,563,020.

L. A molecule that stimulates signal transduction. For example, see the disclosure by Botella, et al., Plant Molec. Biol., 24:757 (1994), of nucleotide sequences for mung bean calmodulin cDNA clones, and Griess, et al., Plant Physiol., 104:1467 (1994), who provide the nucleotide sequence of a maize calmodulin cDNA clone.

M. A hydrophobic moment peptide. See, PCT Application WO 95/16776 and U.S. Pat. No. 5,580,852, which disclose peptide derivatives of tachyplesin which inhibit fungal plant pathogens, and PCT Application WO 95/18855 and U.S. Pat. No. 5,607,914 which teaches synthetic antimicrobial peptides that confer disease resistance.

N. A membrane permease, a channel former or a channel blocker. For example, see the disclosure of Jaynes, et al., Plant Sci, 89:43 (1993), of heterologous expression of a cecropin-β lytic peptide analog to render transgenic tobacco plants resistant to Pseudomonas solanacearum.

O. A viral-invasive protein or a complex toxin derived there from. For example, the accumulation of viral coat proteins in transformed plant cells imparts resistance to viral infection and/or disease development effected by the virus from which the coat protein gene is derived, as well as by related viruses. See, Beachy, et al., Ann. Rev. Phytopathol., 28:451 (1990). Coat protein-mediated resistance has been conferred upon transformed plants against alfalfa mosaic virus, cucumber mosaic virus, and tobacco mosaic virus.

P. An insect-specific antibody or an immunotoxin derived therefrom. Thus, an antibody targeted to a critical metabolic function in the insect gut would inactivate an affected enzyme, killing the insect. See, Taylor, et al., Abstract #497, Seventh Int'l Symposium on Molecular Plant-Microbe Interactions (Edinburgh, Scotland 1994) (enzymatic inactivation in transgenic tobacco via production of single-chain antibody fragments).

Q. A virus-specific antibody. See, for example, Tavladoraki, et al., Nature, 366:469 (1993), who show that transgenic plants expressing recombinant antibody genes are protected from virus attack.

R. A developmental-arrestive protein produced in nature by a pathogen or a parasite. Thus, fungal endo-α-1,4-D-polygalacturonases facilitate fungal colonization and plant nutrient release by solubilizing plant cell wall homo-α-1,4-D-galacturonase. See, Lamb, et al., Bio/Technology, 10:1436 (1992). The cloning and characterization of a gene which encodes a bean endopolygalacturonase-inhibiting protein is described by Toubart, et al., Plant J., 2:367 (1992).

S. A developmental-arrestive protein produced in nature by a plant. For example, Logemann, et al., Bio/Technology, 10:305 (1992), have shown that transgenic plants expressing the barley ribosome-inactivating gene have an increased resistance to fungal disease.

T. Genes involved in the Systemic Acquired Resistance (SAR) Response and/or the pathogenesis-related genes. Briggs, S., Current Biology, 5(2) (1995); Pieterse & Van Loon, Curr. Opin. Plant Bio., 7(4):456-64 (2004); and Somssich, Cell, 113(7):815-6 (2003).

U. Antifungal genes. See, Cornelissen and Melchers, Plant Physiol., 101:709-712 (1993); Parijs, et al., Planta, 183:258-264 (1991); and Bushnell, et al., Can. J. of Plant Path., 20(2):137-149 (1998). See also, U.S. Pat. No. 6,875,907.

V. Detoxification genes, such as for fumonisin, beauvericin, moniliformin, and zearalenone and their structurally-related derivatives. See, for example, U.S. Pat. No. 5,792,931.

W. Cystatin and cysteine proteinase inhibitors. See, U.S. Pat. No. 7,205,453.

X. Defensin genes. See, WO 03/000863 and U.S. Pat. No. 6,911,577.

Y. Genes conferring resistance to nematodes, and in particular soybean cyst nematodes. See, e.g., PCT Applications WO 96/30517, WO 93/19181, and WO 03/033651; Urwin, et al., Planta, 204:472-479 (1998); Williamson, Curr Opin Plant Bio., 2(4):327-31 (1999).

Z. Genes that confer resistance to Phytophthora Root Rot, such as the Rps 1, Rps 1-a, Rps 1-b, Rps 1-c, Rps 1-d, Rps 1-e, Rps 1-k, Rps 2, Rps 3-a, Rps 3-b, Rps 3-c, Rps 4, Rps 5, Rps 6, Rps 7, and other Rps genes. See, for example, Shoemaker, et al., Phytophthora Root Rot Resistance Gene Mapping in Soybean, Plant Genome IV Conference, San Diego, Calif. (1995).

AA. Genes that confer resistance to Brown Stem Rot, such as described in U.S. Pat. No. 5,689,035 and incorporated by reference for this purpose.

Any of the above-listed disease or pest resistance genes (A-AA) can be introduced into the claimed soybean cultivar through a variety of means including, but not limited to, transformation and crossing.

2. Genes That Confer Resistance to an Herbicide, for example:

A. An herbicide that inhibits the growing point or meristem, such as an imidazolinone or a sulfonylurea. Exemplary genes in this category code for mutant ALS and AHAS enzyme as described, for example, by Lee, et al., EMBO J., 7:1241 (1988) and Miki, et al., Theor. Appl. Genet., 80:449 (1990), respectively.

B. Glyphosate (resistance conferred by mutant 5-enolpyruvlshikimate-3-phosphate synthase (EPSPS) and aroA genes, respectively) and other phosphono compounds, such as glufosinate (phosphinothricin acetyl transferase (PAT) and Streptomyces hygroscopicus PAT bar genes), pyridinoxy or phenoxy proprionic acids, and cyclohexanediones (ACCase inhibitor-encoding genes). See, for example, U.S. Pat. No. 4,940,835 to Shah, et al., which discloses the nucleotide sequence of a form of EPSPS which can confer glyphosate resistance. U.S. Pat. No. 5,627,061 to Barry, et al., also describes genes encoding EPSPS enzymes. See also, U.S. Pat. Nos. 6,566,587, 6,338,961, 6,248,876, 6,040,497, 5,804,425, 5,633,435, 5,145,783, 4,971,908, 5,312,910, 5,188,642, 4,940,835, 5,866,775, 6,225,114, 6,130,366, 5,310,667, 4,535,060, 4,769,061, 5,633,448, 5,510,471, RE 36,449, RE 37,287, and 5,491,288; and International Publications EP1173580, WO 01/66704, EP1173581, and EP1173582, which are incorporated herein by reference for this purpose. Glyphosate resistance is also imparted to plants that express a gene that encodes a glyphosate oxido-reductase enzyme, as described more fully in U.S. Pat. Nos. 5,776,760 and 5,463,175, which are incorporated herein by reference for this purpose. In addition, glyphosate resistance can be imparted to plants by the over expression of genes encoding glyphosate N-acetyltransferase. See, for example, U.S. application Ser. No. 10/427,692. A DNA molecule encoding a mutant aroA gene can be obtained under ATCC Accession No. 39256, and the nucleotide sequence of the mutant gene is disclosed in U.S. Pat. No. 4,769,061 to Comai. European Patent Appl. No. 0 333 033 to Kumada, et al. and U.S. Pat. No. 4,975,374 to Goodman, et al., discloses nucleotide sequences of glutamine synthetase genes which confer resistance to herbicides such as L-phosphinothricin. The nucleotide sequence of a PAT gene is provided in European Patent Appl. No. 0 242 246 to Leemans, et al. DeGreef, et al., Bio/Technology, 7:61 (1989) describe the production of transgenic plants that express chimeric bar genes coding for phosphinothricin acetyl transferase activity. Exemplary of genes conferring resistance to phenoxy proprionic acids and cyclohexones, such as sethoxydim and haloxyfop are the Acc1-S1, Acc1-S2, and Acc2-S3 genes described by Marshall, et al., Theor. Appl. Genet., 83:435 (1992).

C. An herbicide that inhibits photosynthesis, such as a triazine (psbA and gs+ genes) and a benzonitrile (nitrilase gene). Przibila, et al., Plant Cell, 3:169 (1991), describe the transformation of Chlamydomonas with plasmids encoding mutant psbA genes. Nucleotide sequences for nitrilase genes are disclosed in U.S. Pat. No. 4,810,648 to Stalker and DNA molecules containing these genes are available under ATCC Accession Nos. 53435, 67441, and 67442. Cloning and expression of DNA coding for a glutathione S-transferase is described by Hayes, et al., Biochem. J., 285:173 (1992).

D. Acetohydroxy acid synthase, which has been found to make plants that express this enzyme resistant to multiple types of herbicides, has been introduced into a variety of plants. See, Hattori, et al., Mol. Gen. Genet., 246:419 (1995). Other genes that confer tolerance to herbicides include a gene encoding a chimeric protein of rat cytochrome P4507A1 and yeast NADPH-cytochrome P450 oxidoreductase (Shiota, et al., Plant Physiol., 106:17 (1994)); genes for glutathione reductase and superoxide dismutase (Aono, et al., Plant Cell Physiol., 36:1687 (1995)); and genes for various phosphotransferases (Datta, et al., Plant Mol. Biol., 20:619 (1992)).

E. Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306, 6,282,837, 5,767,373, and International Publication WO 01/12825.

Any of the above listed herbicide genes (A-E) can be introduced into the claimed soybean cultivar through a variety of means including but not limited to transformation and crossing.

3. Genes That Confer or Contribute to a Value-Added Trait, such as:

A. Modified fatty acid metabolism, for example, by transforming a plant with an antisense gene of stearyl-ACP desaturase to increase stearic acid content of the plant. See, Knultzon, et al., Proc. Natl. Acad. Sci. USA, 89:2625 (1992).

B. Decreased phytate content: 1) Introduction of a phytase-encoding gene enhances breakdown of phytate, adding more free phosphate to the transformed plant. For example, see, Van Hartingsveldt, et al., Gene, 127:87 (1993), for a disclosure of the nucleotide sequence of an Aspergillus niger phytase gene. 2) Up-regulation of a gene that reduces phytate content. In maize, this, for example, could be accomplished by cloning and then re-introducing DNA associated with one or more of the alleles, such as the LPA alleles, identified in maize mutants characterized by low levels of phytic acid, such as in Raboy, et al., Maydica, 35:383 (1990), and/or by altering inositol kinase activity as in WO 02/059324, U.S. Publ. No. 2003/000901, WO 03/027243, U.S. Publ. No. 2003/0079247, WO 99/05298, U.S. Pat. No. 6,197,561, U.S. Pat. No. 6,291,224, U.S. Pat. No. 6,391,348, WO 2002/059324, U.S. Publ. No. 2003/0079247, WO 98/45448, W 099/55882, and WO 01/04147.

C. Modified carbohydrate composition effected, for example, by transforming plants with a gene coding for an enzyme that alters the branching pattern of starch, or a gene altering thioredoxin, such as NTR and/or TRX (see, U.S. Pat. No. 6,531,648, which is incorporated by reference for this purpose), and/or a gamma zein knock out or mutant, such as cs27 or TUSC27 or en27 (see, U.S. Pat. No. 6,858,778, and U.S. Publ. Nos. 2005/0160488 and 2005/0204418, which are incorporated by reference for this purpose). See, Shiroza, et al., J. Bacteriol., 170:810 (1988) (nucleotide sequence of Streptococcus mutans fructosyltransferase gene); Steinmetz, et al., Mol. Gen. Genet., 200:220 (1985) (nucleotide sequence of Bacillus subtilis levansucrase gene); Pen, et al., Bio/Technology, 10:292 (1992) (production of transgenic plants that express Bacillus licheniformis alpha-amylase); Elliot, et al., Plant Molec. Biol., 21:515 (1993) (nucleotide sequences of tomato invertase genes); Sogaard, et al., J. Biol. Chem., 268:22480 (1993) (site-directed mutagenesis of barley alpha-amylase gene); Fisher, et al., Plant Physiol., 102:1045 (1993) (maize endosperm starch branching enzyme II); WO 99/10498 (improved digestibility and/or starch extraction through modification of UDP-D-xylose 4-epimerase, Fragile 1 and 2, Ref 1, HCHL, C4H); U.S. Pat. No. 6,232,529 (method of producing high oil seed by modification of starch levels (AGP)). The fatty acid modification genes mentioned above may also be used to affect starch content and/or composition through the interrelationship of the starch and oil pathways.

D. Elevated oleic acid via FAD-2 gene modification and/or decreased linolenic acid via FAD-3 gene modification. See, U.S. Pat. Nos. 6,063,947, 6,323,392, and International Publication WO 93/11245. Linolenic acid is one of the five most abundant fatty acids in soybean seeds. The low oxidative stability of linolenic acid is one reason that soybean oil undergoes partial hydrogenation. When partially hydrogenated, all unsaturated fatty acids form trans fats. Soybeans are the largest source of edible-oils in the U.S. and 40% of soybean oil production is partially hydrogenated. The consumption of trans fats increases the risk of heart disease. Regulations banning trans fats have encouraged the development of low linolenic soybeans. Soybeans containing low linolenic acid percentages create a more stable oil requiring hydrogenation less often. This provides trans fat free alternatives in products such as cooking oil.

E. Altering conjugated linolenic or linoleic acid content, such as in WO 01/12800. Altering LEC1, AGP, Dek1, Superal1, mi1ps, and various Ipa genes, such as Ipa1, Ipa3, hpt, or hggt. See, for example, WO 02/42424, WO 98/22604, WO 03/011015, WO 02/057439, WO 03/011015, U.S. Pat. Nos. 6,423,886, 6,197,561, 6,825,397, 7,157,621, U.S. Publ. No. 2003/0079247, and Rivera-Madrid, R., et al., Proc. Natl. Acad. Sci., 92:5620-5624 (1995).

F. Altered antioxidant content or composition, such as alteration of tocopherol or tocotrienols. See, for example, U.S. Pat. Nos. 6,787,683, 7,154,029, WO 00/68393 (involving the manipulation of antioxidant levels through alteration of a phytl prenyl transferase (ppt)); WO 03/082899 (through alteration of a homogentisate geranyl geranyl transferase (hggt)).

G. Altered essential seed amino acids. See, for example, U.S. Pat. No. 6,127,600 (method of increasing accumulation of essential amino acids in seeds); U.S. Pat. No. 6,080,913 (binary methods of increasing accumulation of essential amino acids in seeds); U.S. Pat. No. 5,990,389 (high lysine); U.S. Pat. No. 5,850,016 (alteration of amino acid compositions in seeds); U.S. Pat. No. 5,885,802 (high methionine); U.S. Pat. No. 5,885,801 (high threonine); U.S. Pat. No. 6,664,445 (plant amino acid biosynthetic enzymes); U.S. Pat. No. 6,459,019 (increased lysine and threonine); U.S. Pat. No. 6,441,274 (plant tryptophan synthase beta subunit); U.S. Pat. No. 6,346,403 (methionine metabolic enzymes); U.S. Pat. No. 5,939,599 (high sulfur); U.S. Pat. No. 5,912,414 (increased methionine); U.S. Pat. No. 5,633,436 (increasing sulfur amino acid content); U.S. Pat. No. 5,559,223 (synthetic storage proteins with defined structure containing programmable levels of essential amino acids for improvement of the nutritional value of plants); U.S. Pat. No. 6,194,638 (hemicellulose); U.S. Pat. No. 7,098,381 (UDPGdH); U.S. Pat. No. 6,194,638 (RGP); U.S. Pat. Nos. 6,399,859, 6,930,225, 7,179,955, and 6,803,498; U.S. Publ. No. 2004/0068767; WO 99/40209 (alteration of amino acid compositions in seeds); WO 99/29882 (methods for altering amino acid content of proteins); WO 98/20133 (proteins with enhanced levels of essential amino acids); WO 98/56935 (plant amino acid biosynthetic enzymes); WO 98/45458 (engineered seed protein having higher percentage of essential amino acids); WO 98/42831 (increased lysine); WO 96/01905 (increased threonine); WO 95/15392 (increased lysine); WO 01/79516; and WO 00/09706 (Ces A: cellulose synthase).

4. Genes that Control Male Sterility:

There are several methods of conferring genetic male sterility available, such as multiple mutant genes at separate locations within the genome that confer male sterility, as disclosed in U.S. Pat. Nos. 4,654,465 and 4,727,219 to Brar, et al., and chromosomal translocations as described by Patterson in U.S. Pat. Nos. 3,861,709 and 3,710,511. In addition to these methods, Albertsen, et al., U.S. Pat. No. 5,432,068, describes a system of nuclear male sterility which includes: identifying a gene which is critical to male fertility; silencing this native gene which is critical to male fertility; removing the native promoter from the essential male fertility gene and replacing it with an inducible promoter; inserting this genetically engineered gene back into the plant; and thus creating a plant that is male sterile because the inducible promoter is not “on” resulting in the male fertility gene not being transcribed. Fertility is restored by inducing, or turning “on,” the promoter, which in turn allows the gene that confers male fertility to be transcribed.

A. Introduction of a deacetylase gene under the control of a tapetum-specific promoter and with the application of the chemical N-Ac-PPT. See, International Publication WO 01/29237.

B. Introduction of various stamen-specific promoters. See, International Publications WO 92/13956 and WO 92/13957.

C. Introduction of the barnase and the barstar genes. See, Paul, et al., Plant Mol. Biol., 19:611-622 (1992).

For additional examples of nuclear male and female sterility systems and genes, see also, U.S. Pat. Nos. 5,859,341, 6,297,426, 5,478,369, 5,824,524, 5,850,014, and 6,265,640, all of which are hereby incorporated by reference.

5. Genes that Create a Site for Site Specific DNA Integration:

This includes the introduction of FRT sites that may be used in the FLP/FRT system and/or Lox sites that may be used in the Cre/Loxp system. See, for example, Lyznik, et al., Site-Specific Recombination for Genetic Engineering in Plants, Plant Cell Rep, 21:925-932 (2003) and WO 99/25821, which are hereby incorporated by reference. Other systems that may be used include the Gin recombinase of phage Mu (Maeser, et al. (1991); Vicki Chandler, The Maize Handbook, Ch. 118 (Springer-Verlag 1994)); the Pin recombinase of E. coli (Enomoto, et al. (1983)); and the R/RS system of the pSR1 plasmid (Araki, et al. (1992)).

6. Genes that Affect Abiotic Stress Resistance:

Genes that affect abiotic stress resistance (including but not limited to flowering, pod and seed development, enhancement of nitrogen utilization efficiency, altered nitrogen responsiveness, drought resistance or tolerance, cold resistance or tolerance, and salt resistance or tolerance) and increased yield under stress. For example, see: WO 00/73475 where water use efficiency is altered through alteration of malate; U.S. Pat. Nos. 5,892,009, 5,965,705, 5,929,305, 5,891,859, 6,417,428, 6,664,446, 6,706,866, 6,717,034, 6,801,104, WO 2000/060089, WO 2001/026459, WO 2001/035725, WO 2001/034726, WO 2001/035727, WO 2001/036444, WO 2001/036597, WO 2001/036598, WO 2002/015675, WO 2002/017430, WO 2002/077185, WO 2002/079403, WO 2003/013227, WO 2003/013228, WO 2003/014327, WO 2004/031349, WO 2004/076638, WO 98/09521, and WO 99/38977 describing genes, including CBF genes and transcription factors effective in mitigating the negative effects of freezing, high salinity, and drought on plants, as well as conferring other positive effects on plant phenotype; U.S. Publ. No. 2004/0148654 and WO 01/36596, where abscisic acid is altered in plants resulting in improved plant phenotype, such as increased yield and/or increased tolerance to abiotic stress; WO 2000/006341, WO 04/090143, U.S. application Ser. No. 10/817,483, and U.S. Pat. No. 6,992,237, where cytokinin expression is modified resulting in plants with increased stress tolerance, such as drought tolerance, and/or increased yield. See also, WO 02/02776, WO 2003/052063, JP 2002281975, U.S. Pat. No. 6,084,153, WO 01/64898, and U.S. Pat. Nos. 6,177,275 and 6,107,547 (enhancement of nitrogen utilization and altered nitrogen responsiveness). For ethylene alteration, see, U.S. Publ. Nos. 2004/0128719, 2003/0166197, and WO 2000/32761. For plant transcription factors or transcriptional regulators of abiotic stress, see, e.g., U.S. Publ. Nos. 2004/0098764 or 2004/0078852.

Other genes and transcription factors that affect plant growth and agronomic traits, such as yield, flowering, plant growth, and/or plant structure, can be introduced or introgressed into plants. See, e.g., WO 97/49811 (LHY), WO 98/56918 (ESD4), WO 97/10339, U.S. Pat. Nos. 6,573,430 (TFL), 6,713,663 (FT), 6,794,560, 6,307,126 (GAI), WO 96/14414 (CON), WO 96/38560, WO 01/21822 (VRN1), WO 00/44918 (VRN2), WO 99/49064 (GI), WO 00/46358 (FRI), WO 97/29123, WO 99/09174 (D8 and Rht), WO 2004/076638, and WO 004/031349 (transcription factors).

### Methods for Soybean Transformation

Numerous methods for plant transformation have been developed including biological and physical plant transformation protocols. See, for example, Miki, et al., “Procedures for Introducing Foreign DNA into Plants,” in Methods in Plant Molecular Biology and Biotechnology, Glick and Thompson Eds., CRC Press, Inc., Boca Raton, pp. 67-88 (1993). In addition, expression vectors and in-vitro culture methods for plant cell or tissue transformation and regeneration of plants are available. See, for example, Gruber, et al., “Vectors for Plant Transformation,” in Methods in Plant Molecular Biology and Biotechnology, Glick and Thompson Eds., CRC Press, Inc., Boca Raton, pp. 89-119 (1993).

A. Agrobacterium-mediated Transformation—One method for introducing an expression vector into plants is based on the natural transformation system of Agrobacterium. See, for example, Horsch, et al., Science, 227:1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant Sci., 10:1 (1991). Descriptions of Agrobacterium vector systems and methods for Agrobacterium-mediated gene transfer are provided by Gruber, et al., supra, Miki, et al., supra, and Moloney, et al., Plant Cell Reports, 8:238 (1989). See also, U.S. Pat. No. 5,563,055 (Townsend and Thomas), issued Oct. 8, 1996.

B. Direct Gene Transfer—Several methods of plant transformation, collectively referred to as direct gene transfer, have been developed as an alternative to Agrobacterium-mediated transformation. A generally applicable method of plant transformation is microprojectile-mediated transformation where DNA is carried on the surface of microprojectiles measuring 1 to 4 μm. The expression vector is introduced into plant tissues with a biolistic device that accelerates the microprojectiles to speeds of 300 to 600 m/s which is sufficient to penetrate plant cell walls and membranes. Sanford, et al., Part. Sci. Technol., 5:27 (1987); Sanford, J. C., Trends Biotech., 6:299 (1988); Klein, et al., Bio/Tech., 6:559-563 (1988); Sanford, J. C., Physiol Plant, 7:206 (1990); Klein, et al., Biotechnology, 10:268 (1992). See also, U.S. Pat. No. 5,015,580 (Christou, et al.), issued May 14, 1991 and U.S. Pat. No. 5,322,783 (Tomes, et al.), issued Jun. 21, 1994.

Another method for physical delivery of DNA to plants is sonication of target cells. Zhang, et al., Bio/Technology, 9:996 (1991). Alternatively, liposome and spheroplast fusion have been used to introduce expression vectors into plants. Deshayes, et al., EMBO J., 4:2731 (1985); Christou, et al., Proc Natl. Acad. Sci. USA, 84:3962 (1987). Direct uptake of DNA into protoplasts using CaCl₂ precipitation, polyvinyl alcohol or poly-L-ornithine has also been reported. Hain, et al., Mol. Gen. Genet., 199:161 (1985) and Draper, et al., Plant Cell Physiol., 23:451 (1982). Electroporation of protoplasts and whole cells and tissues have also been described (Donn, et al., In Abstracts of VIIth International Congress on Plant Cell and Tissue Culture IAPTC, A2-38, p. 53 (1990); D'Halluin, et al., Plant Cell, 4:1495-1505 (1992); and Spencer, et al., Plant Mol. Biol., 24:51-61 (1994)).

Following transformation of soybean target tissues, expression of the above-described selectable marker genes allows for preferential selection of transformed cells, tissues, and/or plants, using regeneration and selection methods well known in the art.

The foregoing methods for transformation would typically be used for producing a transgenic variety. The transgenic variety could then be crossed with another (non-transformed or transformed) variety in order to produce a new transgenic variety. Alternatively, a genetic trait that has been engineered into a particular soybean line using the foregoing transformation techniques could be moved into another line using traditional backcrossing techniques that are well known in the plant breeding arts. For example, a backcrossing approach could be used to move an engineered trait from a public, non-elite variety into an elite variety, or from a variety containing a foreign gene in its genome into a variety or varieties that do not contain that gene. As used herein, “crossing” can refer to a simple x by y cross or the process of backcrossing depending on the context.

### Genetic Marker Profile Through SSR and First Generation Progeny

In addition to phenotypic observations, a plant can also be identified by its genotype. The genotype of a plant can be characterized through a genetic marker profile which can identify plants of the same variety, or a related variety, or be used to determine or validate a pedigree. Genetic marker profiles can be obtained by techniques such as Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs) (which are also referred to as Microsatellites), and SNPs. For example, see, Cregan, et al., “An Integrated Genetic Linkage Map of the Soybean Genome,” Crop Science, 39:1464-1490 (1999) and Berry, et al., “Assessing Probability of Ancestry Using Simple Sequence Repeat Profiles: Applications to Maize Inbred Lines and Soybean Varieties,” Genetics, 165:331-342 (2003), each of which are incorporated by reference herein in their entirety.

Particular markers used for these purposes are not limited to any particular set of markers, but are envisioned to include any type of marker and marker profile which provides a means of distinguishing varieties.

Means of performing genetic marker profiles using SSR polymorphisms are well known in the art. SSRs are genetic markers based on polymorphisms in repeated nucleotide sequences, such as microsatellites. A marker system based on SSRs can be highly informative in linkage analysis relative to other marker systems in that multiple alleles may be present. Another advantage of this type of marker is that, through use of flanking primers, detection of SSRs can be achieved, for example, by the polymerase chain reaction (PCR), thereby eliminating the need for labor-intensive Southern hybridization. The PCR detection is done by use of two oligonucleotide primers flanking the polymorphic segment of repetitive DNA. Repeated cycles of heat denaturation of the DNA followed by annealing of the primers to their complementary sequences at low temperatures, and extension of the annealed primers with DNA polymerase, comprise the major part of the methodology.

Following amplification, markers can be scored by electrophoresis of the amplification products. Scoring of marker genotype is based on the size of the amplified fragment, which may be measured by the number of base pairs of the fragment. While variation in the primer used or in laboratory procedures can affect the reported fragment size, relative values should remain constant regardless of the specific primer or laboratory used. When comparing varieties it is preferable if all SSR profiles are performed in the same lab.

Primers used are publicly available and may be found in the Soybase or Cregan supra. See also, PCT Publication No. WO 99/31964 (Nucleotide Polymorphisms in Soybean); U.S. Pat. No. 6,162,967 (Positional Cloning of Soybean Cyst Nematode Resistance Genes); and U.S. application Ser. No. 09/954,773 (Soybean Sudden Death Syndrome Resistant Soybeans and Methods of Breeding and Identifying Resistant Plants), the disclosure of which are incorporated herein by reference.

While determining the SSR genetic marker profile of the plants described supra, several unique SSR profiles may also be identified which did not appear in either parent of such plant. Such unique SSR profiles may arise during the breeding process from recombination or mutation. A combination of several unique alleles provides a means of identifying a plant variety, an F₁ progeny produced from such variety, and progeny produced from such variety.

### Single-Gene Conversions

When the term “soybean plant” is used in the context of the present invention, this also includes any single gene conversions of that variety. The term single gene converted plant as used herein refers to those soybean plants which are developed by a plant breeding technique called backcrossing wherein essentially all of the desired morphological and physiological characteristics of a variety are recovered in addition to the single gene transferred into the variety via the backcrossing technique. Backcrossing methods can be used with the present invention to improve or introduce a characteristic into the variety. The term “backcrossing” as used herein refers to the repeated crossing of a hybrid progeny back to the recurrent parent, i.e., backcrossing 1, 2, 3, 4, 5, 6, 7, 8, or more times to the recurrent parent. The parental soybean plant that contributes the gene for the desired characteristic is termed the nonrecurrent or donor parent. This terminology refers to the fact that the nonrecurrent parent is used one time in the backcross protocol and therefore does not recur. The parental soybean plant to which the gene or genes from the nonrecurrent parent are transferred is known as the recurrent parent as it is used for several rounds in the backcrossing protocol (Poehlman & Sleper (1994); Fehr, Principles of Cultivar Development, pp. 261-286 (1987)). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a soybean plant is obtained wherein essentially all of the desired morphological and physiological characteristics of the recurrent parent are recovered in the converted plant, in addition to the single transferred gene from the nonrecurrent parent.

The selection of a suitable recurrent parent is an important step for a successful backcrossing procedure. The goal of a backcross protocol is to alter or substitute a single trait or characteristic in the original variety. To accomplish this, a single gene of the recurrent variety is modified or substituted with the desired gene from the nonrecurrent parent, while retaining essentially all of the rest of the desired genetic, and therefore the desired physiological and morphological constitution of the original variety. The choice of the particular nonrecurrent parent will depend on the purpose of the backcross; one of the major purposes is to add some agronomically important trait to the plant. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

Many single gene traits have been identified that are not regularly selected for in the development of a new variety but that can be improved by backcrossing techniques. Single gene traits may or may not be transgenic. Examples of these traits include, but are not limited to, male sterility, waxy starch, herbicide resistance, resistance for bacterial, fungal, or viral disease, insect resistance, male fertility, enhanced nutritional quality, industrial usage, yield stability, and yield enhancement. These genes are generally inherited through the nucleus. Several of these single gene traits are described in U.S. Pat. Nos. 5,959,185, 5,973,234, and 5,977,445, the disclosures of which are specifically hereby incorporated by reference.

### Backcross Conversions Using Soybean Plants of the Present Invention

A backcross conversion of soybean plants of the present invention occurs when DNA sequences are introduced through backcrossing (Hallauer, et al., “Corn Breeding,” Corn and Corn Improvements, No. 18, pp. 463-481 (1988)), with the ultra-low trypsin inhibitor soybean utilized as the recurrent parent. Both naturally occurring and transgenic DNA sequences may be introduced through backcrossing techniques. A backcross conversion may produce a plant with a trait or locus conversion in at least two or more backcrosses, including at least 2 crosses, at least 3 crosses, at least 4 crosses, at least 5 crosses, and the like. Molecular marker assisted breeding or selection may be utilized to reduce the number of backcrosses necessary to achieve the backcross conversion. For example, see, Openshaw, S. J., et al., Marker-assisted Selection in Backcross Breeding, Proceedings Symposium of the Analysis of Molecular Data, Crop Science Society of America, Corvallis, Oreg. (August 1994), where it is demonstrated that a backcross conversion can be made in as few as two backcrosses.

The complexity of the backcross conversion method depends on the type of trait being transferred (single genes or closely linked genes as compared to unlinked genes), the level of expression of the trait, the type of inheritance (cytoplasmic or nuclear), and the types of parents included in the cross. It is understood by those of ordinary skill in the art that for single gene traits that are relatively easy to classify, the backcross method is effective and relatively easy to manage. (See, Hallauer, et al., Corn and Corn Improvement, Sprague and Dudley, Third Ed. (1998)). Desired traits that may be transferred through backcross conversion include, but are not limited to, sterility (nuclear and cytoplasmic), fertility restoration, nutritional enhancements, drought tolerance, nitrogen utilization, altered fatty acid profile, low phytate, industrial enhancements, disease resistance (bacterial, fungal, or viral), insect resistance, and herbicide resistance. In addition, an introgression site itself, such as an FRT site, Lox site, or other site specific integration site, may be inserted by backcrossing and utilized for direct insertion of one or more genes of interest into a specific plant variety. In some embodiments of the invention, the number of loci that may be backcrossed into the ultra-low trypsin inhibitor soybean is at least 1, 2, 3, 4, or 5, and/or no more than 6, 5, 4, 3, or 2. A single locus may contain several transgenes, such as a transgene for disease resistance that, in the same expression vector, also contains a transgene for herbicide resistance. The gene for herbicide resistance may be used as a selectable marker and/or as a phenotypic trait. A single locus conversion of site specific integration system allows for the integration of multiple genes at the converted loci.

The backcross conversion may result from either the transfer of a dominant allele or a recessive allele. Selection of progeny containing the trait of interest is accomplished by direct selection for a trait associated with a dominant allele. Transgenes transferred via backcrossing typically function as a dominant single gene trait and are relatively easy to classify. Selection of progeny for a trait that is transferred via a recessive allele requires growing and selfing the first backcross generation to determine which plants carry the recessive alleles. Recessive traits may require additional progeny testing in successive backcross generations to determine the presence of the locus of interest. The last backcross generation is usually selfed to give pure breeding progeny for the gene(s) being transferred, although a backcross conversion with a stably introgressed trait may also be maintained by further backcrossing to the recurrent parent with selection for the converted trait.

Along with selection for the trait of interest, progeny are selected for the phenotype of the recurrent parent. The backcross is a form of inbreeding, and the features of the recurrent parent are automatically recovered after successive backcrosses. Poehlman, Breeding Field Crops, p. 204 (1987). Poehlman suggests from one to four or more backcrosses, but as noted above, the number of backcrosses necessary can be reduced with the use of molecular markers. Other factors, such as a genetically similar donor parent, may also reduce the number of backcrosses necessary. As noted by Poehlman, backcrossing is easiest for simply inherited, dominant, and easily recognized traits.

One process for adding or modifying a trait or locus in soybean plants of the present invention comprises crossing soybean plants of the present invention with plants of other soybeans that comprise the desired trait or locus, selecting F₁ progeny plants that comprise the desired trait or locus to produce selected F₁ progeny plants, crossing the selected progeny plants with soybean plants of the present invention to produce backcross progeny plants, selecting for backcross progeny plants that have the desired trait or locus and the morphological characteristics of soybean plants of the present invention to produce selected backcross progeny plants, and backcrossing to soybean plants of the present invention three or more times in succession to produce selected fourth or higher backcross progeny plants that comprise said trait or locus. The modified soybean plants of the present invention may be further characterized as having the physiological and morphological characteristics of soybean plants of the present invention listed in Table 1 as determined at the 5% significance level when grown in the same environmental conditions and/or may be characterized by percent similarity or identity to soybean plants of the present invention as determined by SSR markers. The above method may be utilized with fewer backcrosses in appropriate situations, such as when the donor parent is highly related or markers are used in the selection step. Desired traits that may be used include those nucleic acids known in the art, some of which are listed herein, that will affect traits through nucleic acid expression or inhibition. Desired loci include the introgression of FRT, Lox, and other sites for site specific integration, which may also affect a desired trait if a functional nucleic acid is inserted at the integration site.

In addition, the above process and other similar processes described herein may be used to produce first generation progeny soybean seed by adding a step at the end of the process that comprises crossing soybean plants of the present invention with the introgressed trait or locus with a different soybean plant and harvesting the resultant first generation progeny soybean seed.

### Tissue Culture

Further reproduction of the variety can occur by tissue culture and regeneration. Tissue culture of various tissues of soybeans and regeneration of plants therefrom is well known and widely published, or example, as referenced in Komatsuda, T., et al., Crop Sci., 31:333-337 (1991); Stephens, P. A., et al., Theor. Appl. Genet., 82:633-635 (1991); Komatsuda, T., et al., Plant Cell, Tissue and Organ Culture, 28:103-113 (1992); Dhir, S., et al., Plant Cell Reports, 11:285-289 (1992); Pandey, P., et al., Japan J. Breed., 42:1-5 (1992); and Shetty, K., et al., Plant Science, 81:245-251 (1992); as well as U.S. Pat. No. 5,024,944, U.S. Pat. No. 5,008,200. Thus, another aspect of this invention is to provide cells which upon growth and differentiation produce soybean plants having the physiological and morphological characteristics of soybean plants of the present invention.

As used herein, the term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollen, flowers, seeds, pods, petioles, leaves, stems, roots, root tips, anthers, pistils, and the like. Means for preparing and maintaining plant tissue culture are well known in the art. By way of example, a tissue culture comprising organs has been used to produce regenerated plants. U.S. Pat. Nos. 5,959,185, 5,973,234, and 5,977,445 describe certain techniques, the disclosures of which are incorporated herein by reference.

### Using Soybean Plants of the Present Invention to Develop Other Soybean Varieties

Soybean plants of the present invention can also provide a source of breeding material that may be used to develop new soybean varieties. Plant breeding techniques known in the art and used in a soybean plant breeding program include, but are not limited to, recurrent selection, mass selection, bulk selection, mass selection, backcrossing, pedigree breeding, open pollination breeding, restriction fragment length polymorphism enhanced selection, genetic marker enhanced selection, making double haploids, and transformation. Often combinations of these techniques are used. The development of soybean varieties in a plant breeding program requires, in general, the development and evaluation of homozygous varieties. There are many analytical methods available to evaluate a new variety. The oldest and most traditional method of analysis is the observation of phenotypic traits, but genotypic analysis may also be used.

### Additional Breeding Methods

Another aspect of the present invention is directed to methods for producing a soybean plant by crossing a first parent soybean plant with a second parent soybean plant wherein either the first or second parent soybean plant is a soybean plants of the present invention. The other parent may be any other soybean plant, such as a soybean plant that is part of a synthetic or natural population. Any such methods using soybean plants of the present invention are part of this invention: selfing, intercrossing, backcrosses, mass selection, pedigree breeding, bulk selection, hybrid production, crosses to populations, and the like. These methods are well known in the art and some of the more commonly used breeding methods are described below. Descriptions of breeding methods can be found in one of several reference books (e.g., Allard, Principles of Plant Breeding (1960); Simmonds, Principles of Crop Improvement (1979); Sneep, et al. (1979); Fehr, “Breeding Methods for Cultivar Development,” Chapter 7, Soybean Improvement, Production and Uses, 2.sup.nd ed., Wilcox editor (1987)).

The following describes breeding methods that may be used with soybean plants of the present invention in the development of further soybean plants. One such embodiment is a method for developing soybean plants of the present invention in a soybean plant breeding program comprising: obtaining the soybean plant or a part thereof of soybean plants of the present invention, utilizing said plant or plant part as a source of breeding material, and selecting soybean plants of the present invention progeny plant with molecular markers in common with soybean plants of the present invention. Breeding steps that may be used in the soybean plant breeding program include pedigree breeding, backcrossing, mutation breeding, and recurrent selection. In conjunction with these steps, techniques such as RFLP-enhanced selection, genetic marker enhanced selection (for example, SSR markers), and the making of double haploids may be utilized.

One of ordinary skill in the art of plant breeding would know how to evaluate the traits of two plant varieties to determine if there is no significant difference between the two traits expressed by those varieties. For example, see, Fehr and Walt, Principles of Cultivar Development, pp. 261-286 (1987). Thus the invention includes progeny soybean plants of the ultra-low trypsin inhibitor soybean, where the progeny comprise a combination of at least two soybean plants of the present invention traits selected from the group consisting of those listed herein, so that said progeny soybean plant is not significantly different for said traits than soybean plants of the present invention as determined at the 5% significance level when grown in the same environmental conditions. Using techniques described herein, molecular markers may be used to identify said progeny plant as a soybean plants of the present invention progeny plant. Mean trait values may be used to determine whether trait differences are significant, and preferably the traits are measured on plants grown under the same environmental conditions. Once such a variety is developed, its value is substantial since it is important to advance the germplasm base as a whole in order to maintain or improve traits such as yield, disease resistance, pest resistance, and plant performance in extreme environmental conditions.

As used herein, the term “plant” includes plant cells, plant protoplasts, plant cell tissue cultures from which soybean plants can be regenerated, plant calli, plant clumps, and plant cells that are intact in plants or parts of plants, such as embryos, pollen, ovules, flowers, pods, leaves, roots, root tips, anthers, cotyledons, hypocotyls, meristematic cells, stems, pistils, petiole, and the like.

### Pedigree Breeding

Pedigree breeding starts with the crossing of two genotypes, such as soybean plants of the present invention and another soybean variety having one or more desirable characteristics that is lacking or which complements soybean plants of the present invention. If the two original parents do not provide all the desired characteristics, other sources can be included in the breeding population. In the pedigree method, superior plants are selfed and selected in successive filial generations. In the succeeding filial generations, the heterozygous condition gives way to homogeneous varieties as a result of self-pollination and selection. Typically in the pedigree method of breeding, five or more successive filial generations of selfing and selection is practiced: F₁ to F₂; F₂ to F₃; F₃ to F₄; F₄ to F₅; etc. After a sufficient amount of inbreeding, successive filial generations will serve to increase seed of the developed variety. Preferably, the developed variety comprises homozygous alleles at about 95% or more of its loci.

In addition to being used to create a backcross conversion, backcrossing can also be used in combination with pedigree breeding. As discussed previously, backcrossing can be used to transfer one or more specifically desirable traits from one variety, the donor parent, to a developed variety called the recurrent parent, which has overall good agronomic characteristics yet lacks that desirable trait or traits. However, the same procedure can be used to move the progeny toward the genotype of the recurrent parent, but at the same time retain many components of the nonrecurrent parent by stopping the backcrossing at an early stage and proceeding with selfing and selection. For example, a soybean variety may be crossed with another variety to produce a first generation progeny plant. The first generation progeny plant may then be backcrossed to one of its parent varieties to create a BC₁ or BC₂. Progeny are selfed and selected so that the newly developed variety has many of the attributes of the recurrent parent and yet several of the desired attributes of the nonrecurrent parent. This approach leverages the value and strengths of the recurrent parent for use in new soybean varieties.

Therefore, an embodiment of this invention is a method of making a backcross conversion of soybean plants of the present invention, comprising the steps of crossing a plant of soybean plants of the present invention with a donor plant comprising a desired trait, selecting an F₁ progeny plant comprising the desired trait, and backcrossing the selected F₁ progeny plant to a plant of soybean plants of the present invention. This method may further comprise the step of obtaining a molecular marker profile of soybean plants of the present invention and using the molecular marker profile to select for a progeny plant with the desired trait and the molecular marker profile of soybean plants of the present invention. In one embodiment, the desired trait is a mutant gene or transgene present in the donor parent.

### Recurrent Selection and Mass Selection

Recurrent selection is a method used in a plant breeding program to improve a population of plants. Soybean plants of the present invention are suitable for use in a recurrent selection program. The method entails individual plants cross pollinating with each other to form progeny. The progeny are grown and the superior progeny selected by any number of selection methods, which include individual plant, half-sib progeny, full-sib progeny, and selfed progeny. The selected progeny are cross pollinated with each other to form progeny for another population. This population is planted and again superior plants are selected to cross pollinate with each other. Recurrent selection is a cyclical process and therefore can be repeated as many times as desired. The objective of recurrent selection is to improve the traits of a population. The improved population can then be used as a source of breeding material to obtain new varieties for commercial or breeding use, including the production of a synthetic cultivar. A synthetic cultivar is the resultant progeny formed by the intercrossing of several selected varieties.

Mass selection is a useful technique when used in conjunction with molecular marker enhanced selection. In mass selection, seeds from individuals are selected based on phenotype or genotype. These selected seeds are then bulked and used to grow the next generation. Bulk selection requires growing a population of plants in a bulk plot, allowing the plants to self-pollinate, harvesting the seed in bulk, and then using a sample of the seed harvested in bulk to plant the next generation. Also, instead of self pollination, directed pollination could be used as part of the breeding program.

### Mutation Breeding

Mutation breeding is another method of introducing new traits into soybean plants of the present invention. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation; such as X-rays, Gamma rays (e.g., cobalt 60 or cesium 137), neutrons, (product of nuclear fission by uranium 235 in an atomic reactor), Beta radiation (emitted from radioisotopes such as phosphorus 32 or carbon 14), or ultraviolet radiation (preferably from 2500 to 2900 nm), or chemical mutagens (such as base analogues (5-bromo-uracil)), related compounds (8-ethoxy caffeine), antibiotics (streptonigrin), alkylating agents (sulfur mustards, nitrogen mustards, epoxides, ethylenamines, sulfates, sulfonates, sulfones, lactones), azide, hydroxylamine, nitrous acid, or acridines. Once a desired trait is observed through mutagenesis the trait may then be incorporated into existing germplasm by traditional breeding techniques. Details of mutation breeding can be found in Fehr, “Principles of Cultivar Development,” Macmillan Publishing Company (1993). In addition, mutations created in other soybean plants may be used to produce a backcross conversion of soybean plants of the present invention that comprises such mutation.

Breeding with Molecular Markers

Molecular markers, which includes markers identified through the use of techniques such as Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARS), Amplified Fragment Length Polymorphisms (AFLPs), Simple Sequence Repeats (SSRs), and Single Nucleotide Polymorphisms (SNPs), may be used in plant breeding methods utilizing soybean plants of the present invention.

Isozyme Electrophoresis and RFLPs have been widely used to determine genetic composition. Shoemaker and Olsen, Molecular Linkage Map of Soybean (Glycine max L. Merr.), pp. 6.131-6.138 (1993). In S. J. O'Brien (ed.), Genetic Maps: Locus Maps of Complex Genomes, Cold Spring Harbor Laboratory Press, Cold Spring Harbor, N.Y., developed a molecular genetic linkage map that consisted of 25 linkage groups with about 365 RFLP, 11 RAPD (random amplified polymorphic DNA), 3 classical markers, and 4 isozyme loci. See also, Shoemaker, R. C., 1994 RFLP Map of Soybean, pp. 299-309; In R. L. Phillips and I. K. Vasil (ed.), DNA-based markers in plants, Kluwer Academic Press Dordrecht, the Netherlands.

SSR technology is currently the most efficient and practical marker technology. More marker loci can be routinely used, and more alleles per marker locus can be found, using SSRs in comparison to RFLPs. For example, Diwan and Cregan described a highly polymorphic microsatellite loci in soybean with as many as 26 alleles. (Diwan, N., and Cregan. P. B., Automated sizing of fluorescent-labeled simple sequence repeat (SSR) markers to assay genetic variation in Soybean, Theor. Appl. Genet., 95:220-225 (1997). SNPs may also be used to identify the unique genetic composition of the invention and progeny varieties retaining that unique genetic composition. Various molecular marker techniques may be used in combination to enhance overall resolution.

Soybean DNA molecular marker linkage maps have been rapidly constructed and widely implemented in genetic studies. One such study is described in Cregan, et al., “An Integrated Genetic Linkage Map of the Soybean Genome,” Crop Science, 39:1464-1490 (1999). Sequences and PCR conditions of SSR Loci in Soybean, as well as the most current genetic map, may be found in Soybase on the World Wide Web.

One use of molecular markers is Quantitative Trait Loci (QTL) mapping. QTL mapping is the use of markers, which are known to be closely linked to alleles that have measurable effects on a quantitative trait. Selection in the breeding process is based upon the accumulation of markers linked to the positive effecting alleles and/or the elimination of the markers linked to the negative effecting alleles from the plant's genome.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select for the genome of the recurrent parent and against the genome of the donor parent. Using this procedure can minimize the amount of genome from the donor parent that remains in the selected plants. It can also be used to reduce the number of crosses back to the recurrent parent needed in a backcrossing program. The use of molecular markers in the selection process is often called genetic marker enhanced selection. Molecular markers may also be used to identify and exclude certain sources of germplasm as parental varieties or ancestors of a plant by providing a means of tracking genetic profiles through crosses.

### Production of Double Haploids

The production of double haploids can also be used for the development of plants with a homozygous phenotype in the breeding program. For example, a soybean plant for which soybean plants of the present invention is a parent can be used to produce double haploid plants. Double haploids are produced by the doubling of a set of chromosomes (1 N) from a heterozygous plant to produce a completely homozygous individual. For example, see, Wan, et al., “Efficient Production of Doubled Haploid Plants Through Colchicine Treatment of Anther-Derived Maize Callus,” Theoretical and Applied Genetics, 77:889-892 (1989) and U.S. Pat. No. 7,135,615. This can be advantageous because the process omits the generations of selfing needed to obtain a homozygous plant from a heterozygous source.

Haploid induction systems have been developed for various plants to produce haploid tissues, plants and seeds. The haploid induction system can produce haploid plants from any genotype by crossing a selected line (as female) with an inducer line. Such inducer lines for maize include Stock 6 (Coe, Am. Nat., 93:381-382 (1959); Sharkar and Coe, Genetics, 54:453-464 (1966); KEMS (Deimling, Roeber, and Geiger, Vortr. Pflanzenzuchtg, 38:203-224 (1997); or KMS and ZMS (Chalyk, Bylich & Chebotar, MNL, 68:47 (1994); Chalyk & Chebotar, Plant Breeding, 119:363-364 (2000)); and indeterminate gametophyte (ig) mutation (Kermicle, Science, 166:1422-1424 (1969). The disclosures of which are incorporated herein by reference.

Methods for obtaining haploid plants are also disclosed in Kobayashi, M., et al., Journ. of Heredity, 71(1):9-14 (1980); Pollacsek, M., Agronomic (Paris) 12(3):247-251 (1992); Cho-Un-Haing, et al., Journ. of Plant Biol., 39(3):185-188 (1996); Verdoodt, L., et al., 96(2):294-300 (February 1998); Genetic Manipulation in Plant Breeding, Proceedings International Symposium Organized by EUCARPIA, Berlin, Germany (Sep. 8-13, 1985); Chalyk, et al., Maize Genet Coop., Newsletter 68:47 (1994).

Descriptions of other breeding methods that are commonly used for different traits and crops can be found in one of several reference books (e.g., Allard (1960); Simmonds (1979); Sneep, et al. (1979); Fehr (1987)).

The use of the terms “a,” “an,” and “the,” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### DEPOSIT INFORMATION

A deposit of the Schillinger Genetics proprietary soybean seed containing the SG-ULTI mutant allele and the Kunitz allele disclosed above and recited in the appended claims has been made with the American Type Culture Collection (ATCC), 10801 University Boulevard, Manassas, Va. 20110. The date of deposit was Feb. 24, 2010. The deposit of 2,500 seeds was taken from the same deposit maintained by Schillinger Genetics since prior to the filing date of this application. All restrictions upon the deposit will be removed upon granting of a patent, and the deposit is intended to meet all of the requirements of 37 C.F.R. §§1.801-1.809. The ATCC accession number is PTA-10684. The deposit will be maintained in the depository for a period of thirty years, or five years after the last request, or for the effective life of the patent, whichever is longer, and will be replaced as necessary during that period.

What is claimed is:

1. A soybean seed having SG-ULTI phenotype of reduced trypsin inhibitor activity, wherein said phenotype is conferred by a presence of both the SG-ULTI mutant allele and a Kunitz allele.

2. A plant, or a part thereof, produced by growing the seed of claim 1.

3. The soybean seed of claim 1, wherein said seed trypsin inhibitor activity is less than 50% of the seed trypsin inhibitor activity of commercial soybean seed.

4. The soybean seed of claim 1, wherein the seed trypsin inhibitor activity is between 25% to 8% of the seed trypsin inhibitor activity of commercial soybean seed.

5. A soybean seed containing the SG-ULTI mutant allele, wherein a representative sample of soybean seed containing the SG-ULTI mutant allele was deposited under ATCC Accession No. PTA-10684.

6. A soybean plant, or a part thereof, produced by growing the seed of claim 5.

7. A soybean plant containing the SG-ULTI mutant allele, wherein a representative sample of soybean seed containing the SG-ULTI mutant allele was deposited under ATCC Accession No. PTA-10684.

8. The soybean plant of claim 2, wherein said plant contains one or more transgenes.

9. The soybean plant of claim 8, wherein said one or more transgenes confers a trait selected from the group comprising herbicide tolerance, disease resistance, insect resistance, pest resistance, altered fatty acid content, altered protein content, altered carbohydrate metabolism, increased grain yield, altered plant maturity, and altered morphological characteristics.

10. A method of producing an herbicide resistant soybean plant, wherein said method comprises introducing a transgene conferring glyphosate herbicide resistance into the plant of claim 2.

11. A tissue culture produced from protoplasts or cells from the plant of claim 2, wherein said cells or protoplasts are produced from a plant part selected from the group consisting of leaf, pollen, ovule, embryo, cotyledon, hypocotyl, meristematic cell, root, root tip, pistil, anther, flower, seed, shoot, stem, pod and petiole.

12. A soybean plant regenerated from the tissue culture of claim 11.

13. A method of producing a soybean seed containing reduced trypsin inhibitor activity, wherein the method comprises:
a. crossing two soybean plants and harvesting the resultant soybean seed, wherein one soybean plant has the SG-ULTI allele and the other soybean plant has the Kunitz allele;
b. growing said seed to produce progeny soybean plants;
c. selecting at least a first progeny plant having reduced trypsin inhibitor activity; and
d. assaying said progeny soybean plants for ultra-low trypsin inhibitor activity.

14. A soybean plant produced by the method of claim 13.

15. A method of producing a soybean food product or feed commodity product comprising obtaining the plant of claim 2 and producing a food product or feed product therefrom.

16. A method of producing a soybean food or feed commodity product comprising obtaining the seed of claim 1 and producing a food product or feed product therefrom.

17. The method of claim 16, wherein the food product or feed commodity product is comprised of a protein concentrate, protein isolate, soybean hulls, meal or flour.

18. The method of claim 16, wherein the food product is oil.

19. The method of claim 16, wherein the food product is comprised of beverages, infused foods, sauces, condiments, salad dressings, fruit juices, syrups, desserts, icings and fillings, soft frozen products, confections or intermediate food.

20. A method of producing soybean seed, comprising crossing the plant of claim 2 with itself or a second soybean plant.

21. A method of preparing hybrid soybean seed, comprised of crossing the plant of claim 2 with a second, different soybean plant.

22. A method of detecting a SG-ULTI mutant allele in a soybean plant or seed, wherein the method comprises:
a. obtaining said soybean plant or seed; and
b. assaying said soybean plant or seed using PCR, hybridization with a labeled nucleotide probe, or DNA sequencing to identify the locus of the SG-ULTI mutant allele.

23. A method of detecting a SG-ULTI mutant allele in a soybean plant or seed, wherein the method comprises:
a. obtaining said soybean plant or seed;
b. assaying said soybean plant or seed for level of trypsin inhibitor activity; and
c. determining if said trypsin inhibitor activity is lower than a seed having a Kunitz allele.

24. The soybean seed of claim 1, wherein the seed trypsin inhibitor activity is between 4,000 TIU to 12,000 TIU.

25. The soybean seed of claim 1, wherein the seed trypsin inhibitor activity is between 4,700 TIU to 11,500 TIU.
